(19) United States
(12) Patent Application Publication
(10) Pub. No.: US 2004/0071666 A1
(43) Pub. Date: Apr. 15, 2004

(54) Compositions and methods related to graft-versus-host disease

(75) Inventors: James L. Ferrara, Ann Arbor, MI (US); Kenneth R. Cooke, Ann Arbor, MI (US); Takanori Teshima, Okayama (JP)

Correspondence Address: MEDLEN & CARROLL, LLP, 101 Howard Street, Suite 350, San Francisco, CA, 94105, US

(73) Assignee: The Regents of the University of Michigan, Ann Arbor, MI

(21) Appl. No.: 10/444,198

(22) Filed: May 23, 2003

Related U.S. Application Data
(60) Provisional application No. 60/383,352, filed on May 24, 2002.

(51) Int. Cl. A61K 45/00

(52) U.S. Cl. 424/93.7

(57) ABSTRACT

The present invention relates to compositions and methods for the treatment and management of graft-versus-host disease and other diseases. In some embodiments, the present invention provides therapies comprising treating subjects with agents that inhibit TNF-α and IL-1.

The present invention claims priority to U.S. Prov. Appln. Ser. No. 60/383,352, filed May 24, 2002, the disclosure of which is incorporated herein in its entirety.

This invention was made in part during work partially supported by the National Institutes of Health, grants CA39542 to Dr. J. L. M. Ferrara and HL03565-05 to Dr. K. R. Cooke.

### FIELD OF THE INVENTION

The present invention relates to compositions and methods for the treatment and management of graft-versus-host disease and other diseases. In some embodiments, the present invention provides therapies comprising treating subjects with agents that inhibit cytokine activity, including, but not limited to, TNF-α and IL-1 activity.

### BACKGROUND OF THE INVENTION

Allogeneic bone marrow transplantation (BMT), an important therapy for a number of hematologic diseases, is complicated by graft-versus-host disease (GVHD). In its acute phase, GVHD is manifest as an inflammatory process, with activation of proinflammatory cytokine cascades, immune effector cells, and target tissue damage (Antin and Ferrara, Blood, 80:2964-2968 (1992); Ferrara, J. Hematother. Stem Cell Res., 9:299-306 (2000)). Multiple cellular populations and cytokines interact in a complex process that ultimately results in apoptotic injury in target organs (skin, gut, liver) and systemic disease. During acute GVHD, activated donor T cells secrete Th1 cytokines, including IFN-γ, in response to host antigens (Rus et al., J. Immunol., 155:2396-2406 (1995)); Kichian et al., J. Immunol., 157:2851-2856 (1996)). Shlomchik and colleagues have shown that host-derived antigen-presenting cells (APCs) play a key role in the initiation of acute GVHD (Sclomchik, et al., Science, 285:412-415). Host APC activation of donor T cells leads to their proliferation and differentiation into effector T cells. Thus, it is thought that TCR and MHC interactions are also required in the effector phase of GVHD, because alloantigen specific cytotoxic T lymphocytes (CTLs) contribute to experimental GVHD (Baker et al., J. Exp. Med., 183:2645-2656 (1996); Braun, et al., J. Exp. Med. 183:657-661 (1996).

Older bone marrow transplantation (BMT) recipients are at heightened risk for acute graft-versus-host disease (GVHD) after allogeneic BMT, but the causes of this association are poorly understood. Advanced age of the BMT recipient is an important determinant of GVHD severity. In one Seattle study, GVHD (grades II-IV) was 20% in patients less than 20 years of age, 30% in patients between 30-45 years of age, and 79% in patients greater than 50 years of age (Sullivan et al., N. Engl. J., 320:828-834 (1989)). The increased risk of GVHD, together with the toxicity of conditioning among patients greater than 60 years old, often excludes older patients from consideration for allogeneic BMT. The mechanism for this association is still poorly understood, although several possibilities have been suggested, including thymic involution and impaired ability of host defenses, increased bacterial and viral colonization of the gastrointestinal (GI) tract, and declining repair processes in damaged tissues with age.

In addition, large numbers of patients with hematologic malignancies are in their seventh decade, and many of these diseases might be treated effectively by allogeneic BMT, but patients of this age are not usually considered for such therapy because of the high risk of GVHD and other treatment-related toxicities (Sullivan et al., N. Engl. J. Med., 320:828-834 (1989); Trimble, et al., Cancer, 74:2208-2214 (1994); Kernan, et al., N. Engl. J. Med., 328:593-602(1993)). A better understanding of GVHD pathophysiology in aged recipients and the role of APCs in this process is needed to improved strategies to reduce this intractable problem and thereby make allogeneic BMT available to the large number of older patients who might benefit from this therapy.

### SUMMARY OF THE INVENTION

The present invention provides methods of transplanting hematopoietic stem cells in a subject diagnosed with a hematopoietic disease comprising the steps of: providing: a subject diagnosed with a hematopoietic disease, hematopoietic stem cells, one or more agents that inhibit one or more cytokines (e.g., an agent that inhibits TNF-α, and an agent that inhibits IL-1, an agent that inhibits IL-6); transplanting the hematopoietic stem cells into the subject to produce a hematopoietic transplant subject; and administering the agent that inhibits the cytokine(s) to the hematopoietic transplant subject. In some embodiments, prior to the transplanting step, the subject is irradiated with a myeloablative dose of radiation. Other embodiments further comprise administering the agent that inhibits the cytokine(s) to the subject prior to transplanting the hematopoietic stem cells. In preferred embodiments, the hematopoietic disease comprises a hematopoietic malignancy selected from the group consisting of leukemia, myelodysplastic syndrome, lymphoma, and plasma cell dyscrasia. In other preferred embodiments, the hematopoietic stem cells are allogeneic hematopoietic stem cells, while in related embodiments the allogeneic hematopoietic stem cells are from a donor related to the subject. In particularly preferred embodiments, the hematopoietic stem cells are selected from the group consisting of bone marrow stem cells, peripheral blood stem cells and umbilical cord blood stem cells. Additionally, in some embodiments the agent that inhibits the cytokine(s) is a cytokine receptor (e.g., a recombinant soluble TNF receptor), an antibody (e.g., an IL-1R-reactive antibody), a small molecule drug inhibitor, an antisense molecule (e.g., an antisense mRNA, an siRNA, a transcription factor decoy, etc.), or any other agent that directly or indirectly inhibits cytokine activity (e.g., IL-1, IL6, or TNF-α activity).

The present invention further provides methods of treating acute graft versus host disease, comprising the steps of: providing a subject with acute graft versus host disease, one or more agents that inhibit one or more cytokines; and administering the agent that inhibits the cytokines to the subject with acute graft versus host disease. In some embodiments, the administering comprises a regimen effective for reducing serum TNF-α levels of the subject. In some embodiments, the administering comprises a regimen effective for reducing serum IL-1β levels of the subject. In preferred embodiments, the administering comprises a regimen effective for increasing the length of post transplant survival of the subject. In other preferred embodiments, the administering comprises a regimen effective for reducing clinical graft versus host disease grades. In some embodiments, the administering comprises a regimen effective for reducing skin pathology of the subject, while in related embodiments the reducing skin pathology comprises maculopapular rash reduction. In other embodiments, the administering comprises a regimen effective for reducing liver pathology of the subject, while in related embodiments the reducing liver pathology comprises reducing elevated serum bilirubin levels. In some embodiments, the administering comprises a regimen effective for reducing intestinal pathology of the subject, while in related embodiments the reducing intestinal pathology comprises reducing diarrhea.

Also provided by the present invention are methods of treating pulmonary dysfunction occurring after allogeneic stem cell transplantation, comprising the steps of: providing a subject diagnosed with pulmonary dysfunction, wherein the pulmonary dysfunction is associated with prior allogeneic stem cell transplantation, one or more agents that inhibit one or more cytokines; and administering the agent that inhibits the cytokine(s) to the subject diagnosed with pulmonary dysfunction. In preferred embodiments, the pulmonary dysfunction is the result of a noninfectious lung injury. In some embodiments, the pulmonary dysfunction comprises a disease including, but not limited to, bronchiolitis obliterans, restrictive lung disease and idiopathic pneumonia syndrome. In preferred embodiments, an agent that inhibits TNF-α comprises a recombinant soluble TNF receptor, while an agent that inhibits IL-1 comprises an IL-1R-reactive antibody. In particularly preferred embodiments, the administering comprises a regimen effective for improving the results of at least one pulmonary function test of the subject. In related embodiments, the at least one pulmonary function test comprises a test selected from the group consisting of a forced vital capacity test (FVC), a forced expiratory volume in one second test (FEV_{1.0}) and a diffuse capacity of lungs for carbon monoxide test (DLCO).

### DESCRIPTION OF THE FIGURES

The following figures form part of the specification and are included to further demonstrate certain aspects and embodiments of the present invention. The invention may be better understood by reference to one or more of these figures in combination with the detailed description of specific embodiments presented herein.

FIG. 1 illustrates the reduced survival and increased severity of GVHD in older recipients after allogeneic bone marrow transplantation. Panel A depicts the survival of B6D2F1 recipients of B6.Ly5.2 bone marrow. Panel B depicts the survival of CBA recipients of B10.BR bone marrow.

FIG. 2 shows that age exacerbates GVHD-associated gastrointestinal tract pathology and serum LPS and TNF-α levels. The small bowel histology: of old recipients of syngeneic bone marrow is shown in panel A, of young recipients of allogeneic bone marrow is shown in panel B, and of old recipients of allogeneic bone marrow is shown in panel C. Panel D shows that old allogeneic recipients have high scores on a GVHD pathology index. Old allogeneic recipients have elevated serum LPS levels (panel E), and elevated serum TNF-α levels (panel F).

FIG. 3 shows that donor T cell responses increase with recipient age. Panel A shows an increase in total T cell numbers, while panel B shows an increase in IFNγ-producing T cell numbers.

FIG. 4 illustrates that CD4+ donor T cells induce GVHD mortality in old allogeneic recipients.

FIG. 5 shows that old antigen presenting cells (APCs) induce increased T cell responses. Panel A shows that greater numbers of CD4+ T cells are found in old recipients. Old APCs: induce greater proliferative responses from young T cells (panel B), increased IFN-γ production from young T cells (panel C), and greater IL-2 production from young T cells (panel D). Old bone marrow-derived dendritic cells (DCs): induce greater proliferative responses from T cells (panel E), produce more TNFα (panel F), and produce more IL-12 (panel G).

FIG. 6 illustrates the increased severity of GVHD in old APC chimeric mice. Panel A shows the reduced survival rate of old APC chimeras, panel B shows the increased clinical GVHD scores of old APC chimeras, and panel C shows the elevated number of donor T cells harvested from the spleens of old APC chimeras.

FIG. 7 shows that MHC class II null mice are resistant to CD4-mediated GVHD. Panel A shows greater cell division in T cells from allogeneic transplants (middle histogram), than is observed in T cells from syngeneic transplants (left histogram), and in T cells from allogeneic transplants from MHC class II null recipients (right histogram). Panel B shows greater expansion in CD4+ T cell numbers in allogeneic recipients, accompanied by increased CD25 and CD49 expression (panel C), and increased IFN-γ production (panel D). Panel E indicates that MHC class II null mice do not succumb to fatal GVHD after allogeneic bone marrow transplantation.

FIG. 8 illustrates that host APCs are sufficient to stimulate allogeneic CD4+ T cells. Panel A shows that host APCs induce allogeneic T cells to divide. Panel B shows that host APCs expand greater numbers of CD4+ T cells, while panel C shows that host APCs induce elevated serum IFN-γ levels.

FIG. 9 illustrates that CD4-mediated GVHD does not require MHC class II alloantigen expression on host epithelial cells. Panel A shows that fatal GVHD occurs even in recipients lacking epithelial MHC class II expression. Panel B shows GVHD-associated liver and small intestine pathology occurs in recipients lacking epithelial MHC class II expression. Panels C and E show histopathology of the liver and small intestine respectively, observed in syngeneic MHC class II null recipients. Panels D and F show histopathology of the liver and small intestine respectively, observed in allogeneic MHC class II null recipients. Panel G shows that alloantigen expression on host APCs is sufficient to elicit fatal GVHD.

FIG. 10 shows that neutralization of inflammatory cytokines prevents CD4+ T cell-mediated GVHD. Alloantigen expression on host APCs results in elevated serum LPS levels (panel A), elevated serum TNFα levels (panel B), and elevated serum IL-1β levels (panel C). Neutralization of TNF-α and IL-1β reduces GVHD mortality (panel D), GVHD clinical score (panel E) and CD4+ donor T cell expansion (panel F). The effect of TNFα and IL-1β neutralization on reduction of GVHD mortality (panel G) and GVHD clinical score (panel H) was reproducible.

FIG. 11 shows that CD8-mediated GVHD does not require MHC class I alloantigen expression on host epithelium. CD8-mediated GVHD may be lethal (panel A) and may be high grade (panel B) in recipients with alloantigen expression on host APCs, even in the absence of alloantigen expression on target cells, and that CD8-mediated GVHD can be reduced by TNF-α and IL-1 neutralization. Panel C shows that CD8-mediated pathology of the liver, intestine and skin occurs in recipients with alloantigen expression on host APCs, while panel D shows that increased numbers of double-positive thymocytes are found in recipients with alloantigen expression on host APCs.

### DEFINITIONS

To facilitate an understanding of the present invention, a number of terms and phrases are defined below:

As used herein, the terms “hematopoietic stem cells” and “haemopoietic stem cells” refer to the progenitor cells of the blood (e.g., red cells, white cells, platelets). The term “hematopoietic stem cells” includes both “multipotent” hematopoietic stem cells that are capable of giving rise to all blood lineages, and “committed” hematopoietic stem cells (derived from multipotent stem cells) that are capable of giving rise to only some blood cell lineages.

The term “hematopoietic graft” as used herein, refers to a portion of living blood used for transplantation purposes. In context of the invention, the term “hematopoietic graft” refers to a graft comprising hematopoietic stem cells.

As used herein, the term “hematopoietic disease” refers to any blood disorder including but not limited to hematopoietic malignancy, hemoglobinopathy, and immunodeficiency.

The term “hematopoietic malignancy” as used herein refers to any blood cell cancer, characterized by uncontrolled, abnormal growth of blood cells. The term “hematopoietic malignancy” includes but is not limited to leukemia, myelodysplastic syndrome, lymphoma, and plasma cell dyscrasia. The term “leukemia” refers to a disease of the blood forming organs characterized by an abnormal increase in the number of leucocytes in the tissues of the body with or without a corresponding increase of those in the circulating blood (e.g., acute lymphoblastic leukemia, ALL; acute myelogenous leukemia, AML; chronic myelogenous leukemia, CML; etc.). The term “myelodysplastic syndrome” refers to a condition in which the bone marrow shows qualitative and quantitative changes suggestive of a preleukaemic process, but having a chronic course that does not necessarily terminate as acute leukaemia. The term “lymphoma” refers to a malignant tumour of lymphoblasts derived from B lymphocytes (e.g., Hodgkin lymphoma, HL; non-Hodgkin lymphoma, NHL; etc.). The term “plasma cell dyscrasia” refers to plasmacytosis due to plasma cell proliferation (e.g., multiple myeloma, MM; plasma cell leukemia, PCL; etc.)

The term “hemoglobinopathy” as used herein refers to disorders involving the oxygen-carrying component of blood known as hemoglobin (e.g., sickle cell anaemia, Fanconi anemia, thalassemia, etc.).

The term “immunodeficiency” as used herein refers to the inability to mount a normal immune response. The term “immunodeficiency” encompasses both inherited (genetic) and acquired immunodeficiencies.

As used herein, the term “subject” refers to organisms to be treated by the methods of the present invention. Such organisms include but are not limited to humans. In the context of the invention, the term “subject” generally refers to an individual who will receive or who has received a hematopoietic graft. In contrast, the term “donor” refers to an individual that supplies living tissue to be used in another body (e.g., a person who furnished blood for transfusion or an organ for transplantation).

The term “diagnosed” as used herein refers to the to recognition of a disease by its signs and symptoms.

As used herein, the term “TNF-α,” “tumor necrosis factor alpha,” and “cachectin” refer to a cytokine that stimulates the recruitment and activation of neutrophils and monocytes. In addition, TNF-α stimulates vascular endothelial cells to express new adhesion molecules, induces macrophages and endothelial cells to secrete chemokines, and promotes apoptosis of target cells. Produced in large amounts, TNFα has systemic effects including induction of fever, synthesis of acute phase proteins by the liver and cachexia (weight loss). As used herein, the term “TNF-β” “tumor necrosis factor beta,” “lymphotoxin,” and “LT” refer to a related proinflammatory factor which binds to the same receptors as TNF-α.

The term “agent that inhibits TNF-α” as used herein refers to any substance capable of repressing or reducing the activity of TNF-α. This term includes but is not limited to neutralizing TNF-α-reactive antibodies, inhibitory soluble TNF-α mutants, soluble TNF receptors, neutralizing TNF receptor-reactive antibodies, antisense molecules, regulators of gene expression, etc. In a preferred embodiment of the present invention, the term “agent that inhibits TNF-α” refers to a soluble recombinant protein known as TNFR-Fc (Etanercept or Enbrel) available from Immunex Corp. (Seattle, Wash.). TNFR-Fc consists of the p75 TNF receptor fused to the gamma immunoglobulin type 1 constant region.

As used herein, the term “TNF receptor” refers to molecules that bind to TNF-α and TNF-β. Currently, two distinct TNF receptors have been identified, TNF-R1 and TNF-RII. TNF-R1 and TNF-RII are members of a large family of homologous receptors with cysteine-rich extracellular domains.

The terms “IL-1,” “interleukin-1,” “catabolin,” and “endogenous pyrogen” refer to a cytokine whose principal function is to mediate host inflammatory responses. There are two forms of IL-1, referred to as IL-1α and IL-1β, that bind to the same receptors and have similar biologic effects, including induction of endothelial cell adhesion molecules, stimulation of chemokine production, stimulation of acute phase reactants, and fever.

As used herein, the term “agent that inhibits IL-1” refers to any substance capable of repressing or reducing the activity of IL-1. This term includes but is not limited to neutralizing IL-1-reactive antibodies, inhibitory soluble IL-1 mutants, soluble IL-1 receptors, neutralizing IL-1 receptor-reactive antibodies, antisense molecules, regulators of gene expression, etc. In a preferred embodiment of the present invention, the term “agent that inhibits IL-1” refers to an antibody reactive with the IL-1 receptor type I.

The terms “IL-1 receptor” and “IL-1R” as used herein refer to molecules that bind to IL-1α and/or IL-1β. Currently, two types of IL-1 receptors have been identified. The IL-1R type I is responsible for mediating the biological activities of IL-1, while the other, the IL-1R type II, is a regulator of the actions of IL-1 by binding IL-1, but failing to deliver a biological signal.

As used herein, the term “mycloablative dose of radiation” and “myeloablative radiation” refer to a dose of radiation sufficient to destroy bone marrow activity. As used herein, the term “myeloablative dose of radiation” is the radiation regimen used to prepare a subject (recipient or patient) for a hematopoietic stem cell graft to both eliminate the subject's diseased bone marrow and to prevent subsequent graft rejection.

The term “allogeneic” as used herein, describes the situation where genes at one or more loci are not identical between two or more individuals. For example, the terms “allogeneic graft” or “allograft” refers to grafts between two or more individuals which differ at one or more loci (usually in reference to major histocompatibility loci). In contrast, the terms “autologous graft” or “autograft” refer to a graft taken from one individual and returned to the same individual. The term “xenograft” refers to a graft taken from an individual of one species and returned to an individual of a different species, genus or family.

As used herein, the term “related” refers to a person connected by blood (e.g., parent, sibling, son, daughter, etc.). In contrast, the term “unrelated” refers to a person not connected by blood (e.g., persons whose sole connection to an individual is as a neighbor, step-parent, in-law, etc). In the context of the invention, hematopoietic grafts from “related” donors are likely to share more loci (and hence may be less likely to cause GVHD) with the intended recipient than would a hematopoietic graft from an “unrelated” donor.

The term “bone marrow” as used herein, refers to the soft, spongy tissue found in the center of most large bones and that produces the cellular components of blood (hematopoiesis).

As used herein, the terms “peripheral blood” “peripheral blood mononuclear cells” “PBMCs” peripheral blood mononuclear cells, “PBLs” refer to white blood cells isolated from a blood sample.

The terms “umbilical cord blood” and “cord blood” as used herein, refer to blood taken from the umbilical cord.

As used herein, the terms “graft versus host disease” and “GVHD” refer to a potentially fatal incompatibility reaction in a subject (host) of low immunological competence that has been the recipient of immunologically competent lymphoid tissue (hematopoietic graft) from a donor who lacks at least one antigen possessed by the recipient/host. GVHD principally affects the gastrointestinal tract, liver, and skin. “Acute GVHD” is generally seen 5-40 days after transplantation, while “chronic GVHD” is generally seen six weeks to several months after transplantation.

The term “serum TNF-α” refers to the amount of TNF-α detected in the liquid portion of the blood after clotting. Similarly, the term “serum IL-1β” refers to the amount of IL-1β detected in the liquid portion of the blood after clotting. In some embodiments, “serum TNF-α” and “serum IL-1β” may be detected by an ELISA.

As used herein, the term “post-transplant survival” refers to the duration of time a subject lives after receiving a hematopoietic graft.

The term “clinical graft versus host disease grade” refers to the grading system used to enumerate the degree of clinical GVHD, and which takes into account clinical changes in skin, liver, and gastrointestinal tract (Glucksberg et al., Transplantation, 18:296-304 (1974); and Flowers et al., Hematology/Oncology Clinics of North America, 13:1091-1112 (1999)).

As used herein, the term “complete response” (CR) refers to complete response of all manifestation of GVHD in that organ. Resolution of manifestations of GVHD occurs within the first 8 weeks of treatment.

As used herein, the term “partial response” (PR) refers to a decrease of at least one grade in the severity of GVHD without deterioration of any organ systems within the first 8 weeks of therapy.

As used herein the term “skin pathology” refers to the skin disorders associated with GVHD. The term “skin pathology” includes but is not limited to any of the following: maculopapular rash, generalized erythroderma, bullous formation and desquamation. The term “maculopapular rash” refers to a series of small, red slightly raised pimples on the skin (measles-like rash).

The term “liver pathology” as used herein refers to the liver disorders associated with GVHD. The term “liver pathology” includes but is not limited to any of the following: jaundice and elevated serum alkaline phosphatase and bilirubin. The term “serum bilirubin level” refers to the level of bilirubin in the serum. In some embodiments, an elevated serum bilirubin level corresponds to 2-3 mg/dl, in other embodiments, an elevated serum bilirubin level corrresponds to 3-6 mg/dl, 6-15 mg/dl or greater than 15 mg/dl.

As used herein, the term “intestinal pathology” refers to gastrointestinal tract disorders associated with GVHD. The term “intestinal pathology” includes but is not limited to any of the following: diarrhea, nausea, anorexia, vomiting, severe abdominal pain, obstruction of the bowel, and bloody stools. The term “diarrhea” refers to abnormally frequent intestinal evacuations with more fluid stools.

The term “pulmonary dysfunction” as used herein refers to disorders of the lung associated with GVHD. The term “pulmonary dysfunction” includes but is not limited to any of the following: noninfectious lung injury, brochiolitis obliterans, restrictive lung disease, and idiopathic pneumonia syndrome.

As used herein, the term “noninfectious lung injury” refers to lung disease observed in the absence of pathogenic bacterial, viral, fungal and mycobacterial microorganisms.

The term “brochiolitis obliterans” refers to a lung disorder characterized by small airway inflammation with fibrinous oliteration of the bronchiolar lumen. In some embodiments of the invention, brochiolitis obliterans is defined by an abnormal pulmonary function test comprising a FEV_{1.0}/FVC test of less than 80% after approximately 3 months post-transplantation, which may be accompanied by cough, dyspnea or wheezing.

The term “restrictive lung disease ” refers to a lung disorder characterized by reductions in lung volume and diffusion capacity. In some embodiments of the invention, restrictive lung disease is defined by an abnormal pulmonary function test comprising a FVC test of less than 80% after approximately 3 months post-transplantation, which may be accompanied by cough, dyspnea or wheezing.

The term “idiopathic pneumonia” refers to a lung disorder characterized by widespread alveolar injury in the absence of active lower respiratory tract infection occurring within approximately 3 months post-transplantation.

As used herein, the term “abnormal pulmonary function” refers to either the need for supplemental oxygen or the presence of infiltrates on radiographic studies of the lung.

The terms “pulmonary function test” and “PFT” as used herein refers to a series of diagnostic studies used to examine the severity of lung disease. The term “PFT” includes but is not limited to a spirogram (obstructive disease gauge), a lung volume determination (restrictive disease gauge), and a diffusion capacity test.

As used herein, the terms “forced vital capacity test” and “FVC” refer to determinations of the volume of air expired with maximal force. It is usually measured along with expiratory flow rates in simple spirometry.

The terms “forced expiratory volume in one second test” and “FEV1.0” as used herein, refer to determinations of the volume of air forcefully expired during the first second after a full breath (normally accounts for >75% of the FVC). This value is recorded both as an absolute value and as a percentage of the FVC (FEV 1% FVC).

As used herein, the terms “diffuse capacity of lungs for carbon monoxide test” “DLCO” refer to determinations of carbon monoxide absorption upon in respiration. For the “DLCO” test, a patient inspires a known small amount of carbon monoxide, holds his breath for ten secconds, then exhales. A sample of alveolar (end-expired) gas is analyzed for carbon monoxide, and the amount absorbed during that breath is then calculated and expressed as mL/min/mm Hg.

As used herein, the term “competes for binding” is used in reference to a first polypeptide with an activity that binds to the same substrate as does a second polypeptide with an activity, where the second polypeptide is a variant of the first polypeptide or a related or dissimilar polypeptide. The efficiency (e.g., kinetics or thermodynamics) of binding by the first polypeptide may be the same as or greater than or less than the efficiency substrate binding by the second polypeptide. For example, the equilibrium binding constant (K_{D}) for binding to the substrate may be different for the two polypeptides. The term “Kₘ” as used herein refers to the Michaelis-Menton constant for an enzyme and is defined as the concentration of the specific substrate at which a given enzyme yields one-half its maximum velocity in an enzyme catalyzed reaction.

As applied to polypeptides, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity or more (e.g., 99 percent sequence identity). Preferably, residue positions that are not identical differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine.

The term “fragment” as used herein refers to a polypeptide that has an amino-terminal and/or carboxy-terminal deletion as compared to the native protein, but where the remaining amino acid sequence is identical to the corresponding positions in the amino acid sequence deduced from a full-length cDNA sequence. Fragments typically are at least 4 amino acids long, preferably at least 20 amino acids long, usually at least 50 amino acids long or longer, and span the portion of the polypeptide required for intermolecular binding of the compositions (claimed in the present invention) with its various ligands and/or substrates.

As used herein, the term “recombinant DNA molecule” as used herein refers to a DNA molecule that is comprised of segments of DNA joined together by means of molecular biological techniques.

As used herein, the term “antisense” is used in reference to RNA sequences that are complementary to a specific RNA sequence (e.g., mRNA). Included within this definition are antisense RNA (“asRNA”) molecules involved in gene regulation by bacteria. Antisense RNA may be produced by any method, including synthesis by splicing the gene(s) of interest in a reverse orientation to a viral promoter that permits the synthesis of a coding strand. Once introduced into an embryo, this transcribed strand combines with natural mRNA produced by the embryo to form duplexes. These duplexes then block either the further transcription of the mRNA or its translation. In this manner, mutant phenotypes may be generated. The term “antisense strand” is used in reference to a nucleic acid strand that is complementary to the “sense” strand. The designation (−) (i.e., “negative”) is sometimes used in reference to the antisense strand, with the designation (+) sometimes used in reference to the sense (i.e., “positive”) strand. Regions of a nucleic acid sequences that are accessible to antisense molecules can be determined using available computer analysis methods.

The term “test compound” refers to any chemical entity, pharmaceutical, drug, and the like that can be used to treat or prevent a disease, illness, sickness, or disorder of bodily function, or otherwise alter the physiological or cellular status of a sample. Test compounds comprise both known and potential therapeutic compounds. A test compound can be determined to be therapeutic by screening using the screening methods of the present invention. A “known therapeutic compound” refers to a therapeutic compound that has been shown (e.g., through animal trials or prior experience with administration to humans) to be effective in such treatment or prevention.

The term “sample” as used herein is used in its broadest sense. A sample suspected of containing a human chromosome or sequences associated with a human chromosome may comprise a cell, chromosomes isolated from a cell (e.g., a spread of metaphase chromosomes), genomic DNA (in solution or bound to a solid support such as for Southern blot analysis), RNA (in solution or bound to a solid support such as for Northern blot analysis), cDNA (in solution or bound to a solid support) and the like. A sample suspected of containing a protein may comprise a cell, a portion of a tissue, an extract containing one or more proteins and the like.

The term “antigenic determinant” as used herein refers to that portion of an antigen that makes contact with a particular antibody (i.e., an epitope). When a protein or fragment of a protein is used to immunize a host animal, numerous regions of the protein may induce the production of antibodies that bind specifically to a given region or three-dimensional structure on the protein; these regions or structures are referred to as antigenic determinants. An antigenic determinant may compete with the intact antigen (i.e., the “immunogen” used to elicit the immune response) for binding to an antibody.

### GENERAL DESCRIPTION OF THE INVENTION

Alloantigen expression on host antigen-presenting cells (APCs) is essential to initiate graft-versus-host disease (GVHD), and alloantigen expression on host target epithelium is, therefore, also thought to be essential for tissue damage. However, during the development of the present invention it was shown that acute GVHD does not require alloantigen expression on host target epithelium, although an understanding of the mechanism is not necessary to practice the present invention and the present invention is not limited to any particular mechanism. Also, during the development of the present invention, it was shown that neutralization of tumor necrosis factor-α (TNF-α) and interleukin-1 (IL-1) prevents acute GVHD. It is also contemplated that neutralization of interleukin-6 (IL-6) prevents acute GVHD. In some embodiments, CD-4 mediated GVHD is prevented with neutralization of tumor necrosis factor-α (TNF-α) and interleukin-1 (IL-1), or related factors. In other embodiments, CD-8 mediated GVHD is prevented with tumor necrosis factor-α (TNF-α) and interleukin-1 (IL-1), or related factors. In still other embodiments, conditions such as multi-organ failure and lung disease, and related conditions are treated. The relationships between T cell subsets (CD4+ or CD8+) and MHC alloantigenic targets are well described (Korngold & Sprent, “T cell subsets in graft-vs.-host disease” in Graft-vs.-Host Disease: Immunology, Pathophysiology, and Treatment, pp. 31-50 (Marcel Dekker, New York, (1990)). An understanding of the mechanism is not required to practice the present invention and the present invention is not limited to any particular mechanism. The present invention demonstrates that when donors and recipients differ only at MHC class II loci, CD4+ cells alone cause lethal GVHD, while CD8+ cells are responsible for GVHD after BMT across only MHC class I difference. Although MHC class I is present on virtually all nucleated cells, MHC class II expression is primarily limited to APCs. During GVHD, MHC class II molecules aberrantly express on epithelial and endothelial target cells (Lampert et al., Nature, 293:149-150 (1981); Mason et al., Nature, 293:150-151 (1981); Barclay and Mason, J. Exp. Med., 156:1665-1676(1982)), and it is generally assumed that this aberrant MHC expression is essential for target cell damage in GVHD. In the present invention, MHC class II or MHC class I alloantigen expression on target cells was tested to determine if alloantigen expression was needed for organ damage of GVHD and subsequent mortality. Embodiments of the invention included using bone marrow (BM) chimeras expressing MHC class II or MHC class I alloantigens only on BM-derived APCs, but not on nonhematopoietic target cells such as epithelium, endothelium, and parenchyma. The present invention also demonstrates that acute GVHD does not require alloantigen expression on host target cells. These results challenge current paradigms about the antigen specificity of GVHD effector mechanisms and demonstrate the central roles of both host APCs and inflammatory cytokines in acute GVHD.

The present invention provides the mediation of GVHD in subjects undergoing a bone marrow transplant. For example, data provided herein demonstrates success of the method in a bone marrow transplant model. Results showed that GVHD mortality, morbidity, and pathologic and biochemical indices were all worse in old recipients. Donor T cell responses were significantly increased in old recipients both in vivo and in vitro when stimulated by antigen-presenting cells from old animals, which also secreted more TNF-α and IL-12 after lipopolysaccharide (LPS) stimulation. CD4+ donor T cells, but not CD8+ T cells, mediated more severe GVHD in old animals. The role of aged APCs in GVHD using bone marrow (BM) animal chimeras created with either old or young BM was confirmed. Four months after animal chimera creation, allogeneic BMT caused significantly worse GVHD in old BM chimeras. APCs from these animals also stimulated greater responses from allogeneic cells in vitro. While an understanding of the mechanism is not required to practice the present invention and the present invention is not limited to any particular mechanism, these data demonstrate a previously unexpected mechanism of amplified donor T cell responses by aged allogeneic host APCs that increases acute GVHD in aged recipients.

It is contemplated that increased GVHD seen in elderly recipients involve the altered function of the immune system of old recipients, increased load of bacterial and viral antigens with age, and reduced repair capacity of aged tissues. The process of thymic involution (decreased thymic size and function) that occurs with increasing age has long been thought to be related to increased GVHD in the elderly (Storb and Thomas, Immunol. Rev., 88:215-238 (1985)). Mackall et al. showed that the capacity to produce new CD4+ T cells expressing CD45RA following chemotherapy is inversely correlated with patient age (Mackall et al., N. Engl. J. Med., 332:143-149(1995)). While an understanding of the mechanism is not required to practice the present invention and the present invention is not limited to any particular mechanism, it is contemplated that decreased thymic function in aged recipients amplifies acute GVHD by reducing host resistance to donor T cell expansion. As a result of thymic involution, the export of naive T cells from the thymus decreases and consequently shifts the ratio of naive T cells to memory T cells. This shift is associated with a decline in proliferation and IL-2 production in response to mitogens (Roberts-Thomson, et al., Lancet, 2:368-370 (1974); Lerner et al., Eur. J. Immunol., 19:977-982 (1989); Utsuyama et al., Mech. Ageing Dev., 63:57-68 (1992)). It is contemplated that this decline in T cell function with age is a type of acquired immunodeficiency that makes older patients more susceptible to a GVH reaction. GVHD itself also damages the thymus, resulting in a loss of negative selection and emergence of host-reactive clones (Van den Brink et al., Transplantation, 69:446-449 (2000)); Hollander, et al., J. Immunol., 152:1609-1617 (1994); Fukushi et al., Proc. Natl. Acad. Sci. USA, 87:6301-6305 (1990)). It is contemplated that this loss of central self-tolerance has important implications for chronic GVHD, but its role in acute GHVD may be less direct.

The present invention demonstrates that APCs from aged mice caused greater allogeneic responses both in vitro and in vivo. Both TNF-α and IL-12 production from dendritic cells (DCs) increased significantly with advancing age after LPS stimulation. TNF-α is known to play an important role in augmenting alloreactive T cell responses (Hill, et al., J. Immunol., 164:656-663 (2000); Kitabayashi et al., Blood, 86:2220-2227 (1995)), and several groups have shown that TNF-α production increases with advancing age of human monocytes and murine macrophages (Rink et al., Mech. Ageing Dev., 102:199-209 (1998); Fagiolo et al., Eur. J. Immunol., 23:2375-2378 (1993); Chorinchath, et al., J. Immunol., 156:1525-1530 (1996)), although information regarding altered function of DCs with aging is scarce (Saurwein-Teissl, et al., Mech. Ageing Dev., 121:123-130 (2000)). IL-12 induces Th1 cytokines and is able to stimulate the development of acute GVHD (Via et al., J. Immunol., 153:4040-4047 (1994); Williamson et al., J. Immunol., 157:689-699 (1996)).

The present invention includes embodiments which demonstrate that for acute GVHD: (1) host APCs are sufficient in both activation and effector phase; (2) alloantigen expression on host target cells is not required, and therefore the effector phase is not antigen specific; and (3) inflammatory cytokines mediate mortality and target destruction. The invention has important clinical implications because donor CTLs are important effectors of beneficial graft-versus-leukemia (GVL) effects. (Truitt et al., Graft versus leukemia. in Graft-vs-Host Disease (eds. Ferrara, J. L. M., Deeg, H. J. & Burakoff, S. J.) 385 (Marcel Dekker, Inc., New York, (1997)). BMT for hematologic malignancies in conjunction with elective blockade of inflammatory cytokines, may be an effective strategy to preserve GVL while reducing toxicity of GVHD.

### DETAILED DESCRIPTION OF THE INVENTION

### I. Conditions Treated by the Present Invention

The present invention finds use in the treatment of conditions including, but not limited to, acute GVHD, CD-4 mediated GVHD, CD-8 mediated GVHD, multi-organ failure associated with BMT, skin pathologies, liver pathologies and lung pathologies associated with BMT. More specifically, the present invention finds use in treating lung pathologies including, but not limited to, idiopathic pneumonia syndrome (IPS), which includes widespread alveolar injury. Also, the present invention finds use in treating and/or preventing obstructive lung disease (bronchiolitis obliterans) and restrictive lung injury associated with BMT. In skin, the invention finds use, for example, for treating maculopapular rash. In the liver, the present invention finds use in reducing serum bilirubin levels.

### II Agents of the Present Invention

The present invention includes agents that modulate cytokines that affect the diseases and conditions described above. Embodiments of the present invention utilize agents that inhibit cytokine activity (e.g., by binding cytokines, their targets, or factors that increase cytokine expression, activity, or bioavailability), including, but not limited to, antibodies, receptors, antisense molecules (e.g., antisense oligonucleotides that target mRNA, siRNAs, and transcription factor decoys that alter the expression of cytokines), small molecule drugs, peptide inhibitors, and other agents that reduce or eliminate cytoknie activity. Preferred specific inhibitors of the present invention include, but are not limited to, TNF-α-reactive antibodies, inhibitory soluble TNF-α mutants, soluble TNF receptors, and neutralizing TNF receptor-reactive antibodies. In a preferred embodiment of the present invention, the soluble recombinant protein known as TNFR-Fc (Etanercept or ENBREL) available from Immunex Corp. (Seattle, Wash.) is used. TNFR-Fc consists of the p75 TNF receptor fused to the gamma immunoglobulin type 1 constant region two distinct TNF receptors have been identified, TNF-R1 and TNF-RII. In another embodiment, Infliximab or Remicade, available from Centocor, Inc (Malvern, Pa.), a chimeric anti-TNF-α monoclonal antibody, which also binds with high affinity to soluble TNF-α is used.

Further embodiments of the invention include, but are not limited to, neutralizing IL-1-reactive antibodies, inhibitory soluble IL-1 mutants, soluble IL-1 receptors, and neutralizing IL-1 receptor-reactive antibodies. In n preferred embodiment of the present invention, an antibody reactive with the IL-1 receptor type I is used. The IL-1R type I is responsible for mediating the biological activities of IL-1, while the other, the IL-1R type II, is a regulator of the actions of IL-1 by binding IL-1. In still further embodiments, compounds that neutralize IL-1 in effect include antagonists to IL-1R, such as but not limited to IL-1Ra, also known as Kineret, is available from Amgen, Inc. (Thousand Oaks, Calif.). Kineret competes with IL-1 for IL-1R sites and in effect neutralizes the action of IL-1.

Another embodiment includes the use of ICEBERG. ICEBERG is a novel protein that inhibits generation of IL-1β by interacting with caspase-1 and preventing its association with RIP2. ProIL-1beta is a proinflammatory cytokine that is proteolytically processed to its active form by caspase-1. Upon receipt of a proinflammatory stimulus, an upstream adaptor, RIP2, binds and oligomerizes caspase-1 zymogen, promoting its autoactivation. (Humke, et al., Cell, 103(1):99-111(2000)). In this embodiment, ICEBERG finds use in turning off IL-1β production which contributes to GVHD.

Other embodiments of the present invention utilize agents including, but not limited to neutralizing IL-6-reactive antibodies, inhibitory soluble IL-6 mutants, soluble IL-6 receptors, and neutralizing IL-6 receptor-reactive antibodies. It is contemplated that neutralization of IL-6, as with IL-1 and TNF-α, finds use as an effective therapy for GVHD. Xun et al., have demonstrated that IL-6 is also up-regulated in response to BMT. (Xun et al., Transplantation,64(2):297-302(1997).

In another embodiment, suberoylanilide hydroxamic acid (SAHA) finds use as a neutralizer of GHVD promoting cytokines. In this particular embodiment, SAHA is a hydroxamic acid-containing hybrid polar molecule that specifically binds to and inhibits the activity of histone deacetylase. (Leoni, et al., Proc. Natl. Acad. Sci. U S A, 99:2995-3000 (2002)). A single oral administration of SAHA to mice dose-dependently reduced circulating TNF-α, IL-1-β, IL-6, and IFN-γ induced by lipopolysaccharide (LPS). Administration of SAHA also reduced hepatic cellular injury in mice following i.v. injection of Con A. (Leoni, et al., Proc. Natl. Acad. Sci. U S A, 99:2995-3000 (2002)). SAHA finds use in down regulating GVHD promoting cytokines, such as, but not limited to, TNF-α, IL-1-β, IL-6, and IFN-γ.

The antibodies discussed supra may be prepared using various immunogens. Embodiments include using the cytokines that promote GVHD themselves or fragments thereof as the immunogen to generate antibodies. In still further embodiments, receptors to GHVD promoting cytokines and fragments thereof may be used as an immunogen to generate antibodies. Such antibodies include, but are not limited to polyclonal, monoclonal, chimeric, single chain, Fab fragments, and Fab expression libraries.

Various procedures known in the art may be used for the production of polyclonal antibodies directed against GVHD promoting cyotkines. For the production of antibody, various host animals can be immunized by injection with the peptide corresponding to an epitope of the GVHD promoting cytokines including but not limited to rabbits, mice, rats, sheep, goats, etc. In a preferred embodiment, the peptide is conjugated to an immunogenic carrier (e.g., diphtheria toxoid, bovine serum albumin (BSA), or keyhole limpet hemocyanin (KLH)). Various adjuvants may be used to increase the immunological response, depending on the host species, including but not limited to Freund's (complete and incomplete), mineral gels (e.g., aluminum hydroxide), surface active substances (e.g., lysolecithin, pluronic polyols, polyanions, peptides, oil emulsions, keyhole limpet hemocyanins, dinitrophenol, and potentially useful human adjuvants such as BCG (Bacille Calmette-Guerin) and Corynebacterium parvum).

For preparation of monoclonal antibodies directed toward GVHD promoting cytokines, it is contemplated that any technique that provides for the production of antibody molecules by continuous cell lines in culture will find use with the present invention (see e.g., Harlow and Lane, Antibodies: A Laboratory Manual, Cold Spring Harbor Laboratory Press, Cold Spring Harbor, N.Y.). These include but are not limited to the hybridoma technique originally developed by Köhler and Milstein (Köhler and Milstein, Nature, 256:495-497 [1975]), as well as the trioma technique, the human B-cell hybridoma technique (see e.g., Kozbor et al., Immunol. Tod., 4:72 [1983]), and the EBV-hybridoma technique to produce human monoclonal antibodies (Cole et al., “The EBV-hybridoma technique and its application to human lung cancer,” in Monoclonal Antibodies and Cancer Therapy, Alan R. Liss, Inc., pp. 77-96 [1985]).

In an additional embodiment of the invention, monoclonal antibodies are produced in germ-free animals utilizing technology such as that described in PCT/US90/02545). Furthermore, it is contemplated that human antibodies will be generated by human hybridomas (Cote et al., Proc. Natl. Acad. Sci. USA, 80:2026-2030 [1983]) or by transforming human B cells with EBV virus in vitro (Cole et al., “The EBV-hybridoma technique and its application to human lung cancer,” in Monoclonal Antibodies and Cancer Therapy, Alan R. Liss, pp. 77-96 [1985]).

In addition, it is contemplated that techniques described for the production of single chain antibodies (U.S. Pat. No. 4,946,778; herein incorporated by reference) will find use in producing GVHD promoting cytokine specific single chain antibodies. An additional embodiment of the invention utilizes the techniques described for the construction of Fab expression libraries (Huse et al., Science, 246:1275-1281 [1989]) to allow rapid and easy identification of monoclonal Fab fragments with the desired specificity.

It is contemplated that any technique suitable for producing antibody fragments will find use in generating antibody fragments that contain the idiotype (antigen binding region) of the antibody molecule. For example, such fragments include but are not limited to: F(ab′)2 fragments that can be produced by pepsin digestion of the antibody molecule; Fab′ fragments that can be generated by reducing the disulfide bridges of the F(ab′)2 fragment, and Fab fragments that can be generated by treating the antibody molecule with papain and a reducing agent.

In the production of antibodies, it is contemplated that screening for the desired antibody will be accomplished by techniques known in the art (e.g., radioimmunoassay, ELISA (enzyme-linked immunosorbant assay), “sandwich” immunoassays, immunoradiometric assays, gel diffusion precipitation reactions, immunodiffusion assays, in situ immunoassays (e.g., using colloidal gold or enzyme or radioisotope labels), Western blots, precipitation reactions, agglutination assays (e.g., gel agglutination assays, hemagglutination assays, etc.), complement fixation assays, immunofluorescence assays, protein A assays, and immunoelectrophoresis assays, etc.

In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention. (As is well known in the art, the immunogenic peptide should be provided free of the carrier molecule used in any immunization protocol. For example, if the peptide was conjugated to KLH, it may be conjugated to BSA, or used directly, in a screening assay.)

Other embodiments of the present invention provide fragments, fusion proteins or functional equivalents of GVHD promoting cytokines or their receptors. In still other embodiments of the present invention, nucleic acid sequences corresponding to these various homologs and mutants may be used to generate recombinant DNA molecules that direct the expression of the homologs and mutants in appropriate host cells. In some embodiments of the present invention, the polypeptide may be a naturally purified product, in other embodiments it may be a product of chemical synthetic procedures, and in still other embodiments it may be produced by recombinant techniques using a prokaryotic or eukaryotic host (e.g., by bacterial, yeast, higher plant, insect and mammalian cells in culture). In some embodiments, depending upon the host employed in a recombinant production procedure, the polypeptide of the present invention may be glycosylated or may be non-glycosylated. In other embodiments, the polypeptides of the invention may also include an initial methionine amino acid residue.

Still, other embodiments of the present invention provide mutant or variant forms of GVHD promoting cytokines or their receptors. It is possible to modify the structure of a peptide of GVHD promoting cytokines or their receptors for such purposes as enhancing therapeutic or prophylactic efficacy, or stability (e.g., ex vivo shelf life, and/or resistance to proteolytic degradation in vivo). Such modified peptides are considered functional equivalents of GVHD promoting cytokines or their receptors as defined herein. A modified peptide can be produced in which the amino acid sequence has been altered, such as by amino acid substitution, deletion, or addition.

This invention further contemplates a method of generating sets of combinatorial mutants of the present GVHD promoting cytokines or their receptors, as well as truncation mutants, and is especially useful for identifying potential variant sequences (i.e., homologs) that are able to bind and compete. The purpose of screening such combinatorial libraries is to generate, for example, novel homologs that can act as either agonists or antagonists, or alternatively, possess novel activities all together.

In other embodiments of the present invention, combinatorially-derived homologs are generated which have a selective potency relative to a naturally occurring GVHD promoting cytokines or their receptors. Such proteins, when expressed from recombinant DNA constructs, can be used in gene therapy protocols.

Still other embodiments of the present invention provide homologs that have intracellular half-lives dramatically different than the corresponding wild-type protein. For example, the altered protein can be rendered either more stable or less stable to proteolytic degradation or other cellular process that result in destruction of, or otherwise compete with their natural counterpart. Such homologs, and the genes which encode them, can be utilized to alter the location of expression by modulating the half-life of the protein. For instance, a short half-life can give rise to more transient biological effects and, when part of an inducible expression system, can allow tighter control of levels within the cell. As above, such proteins, and particularly their recombinant nucleic acid constructs, can be used in gene therapy protocols.

In still other embodiments of the present invention, homologs are generated by the combinatorial approach to act as antagonists, in that they are able to interfere with the ability of the corresponding wild-type protein to regulate cell function. These antagonists may be useful in the controlled production of animal models with dose-dependent manifestations for further study.

The methods of the present invention find use in treating diseases or altering physiological states. Peptides can be administered to the patient intravenously in a pharmaceutically acceptable carrier such as physiological saline. Standard methods for intracellular delivery of peptides can be used (e.g., delivery via liposome). Such methods are well known to those of ordinary skill in the art. The formulations of this invention are useful for parenteral administration, such as intravenous, subcutaneous, intramuscular, and intraperitoneal. Therapeutic administration of a polypeptide intracellularly can also be accomplished using gene therapy as described above.

As is well known in the medical arts, dosages for any one patient depends upon many factors, including the patient's size, body surface area, age, the particular compound to be administered, sex, time and route of administration, general health, and interaction with other drugs being concurrently administered.

In some embodiments, the nucleic acids of GVHD promoting cytokines and/or their receptors and homologs thereof are oriented in a vector and expressed so as to produce antisense transcripts. To accomplish this, a nucleic acid segment from the desired gene is cloned and operably linked to a promoter such that the antisense strand of RNA will be transcribed. The expression cassette is then transformed into cells and the antisense strand of RNA is produced. The nucleic acid segment to be introduced generally will be substantially identical to at least a portion of the endogenous gene or genes to be repressed. The sequence, however, need not be perfectly identical to inhibit expression. The vectors of the present invention can be designed such that the inhibitory effect applies to other proteins within a family of genes exhibiting homology or substantial homology to the target gene.

Accordingly, in some embodiments of the present invention, neutralizing agents can be administered to a patient alone, or in combination with other nucleotide sequences, drugs or hormones or in pharmaceutical compositions where it is mixed with excipient(s) or other pharmaceutically acceptable carriers. In one embodiment of the present invention, the pharmaceutically acceptable carrier is pharmaceutically inert. In another embodiment of the present invention, agents may be administered alone to individuals subject to or suffering from a disease.

Depending on the condition being treated, these pharmaceutical compositions may be formulated and administered systemically or locally. Techniques for formulation and administration may be found in the latest edition of “Remington's Pharmaceutical Sciences” (Mack Publishing Co, Easton, Pa.). Suitable routes may, for example, include oral or transmucosal administration; as well as parenteral delivery, including intramuscular, subcutaneous, intramedullary, intrathecal, intraventricular, intravenous, intraperitoneal, or intranasal administration.

For injection, the pharmaceutical compositions of the invention may be formulated in aqueous solutions, such as in physiologically compatible buffers such as Hanks' solution, Ringer's solution, or physiologically buffered saline. For tissue or cellular administration, penetrants appropriate to the particular barrier to be permeated are used in the formulation. Such penetrants are generally known in the art.

In other embodiments, the pharmaceutical compositions of the present invention can be formulated using pharmaceutically acceptable carriers well known in the art in dosages suitable for oral administration. Such carriers enable the pharmaceutical compositions to be formulated as tablets, pills, capsules, liquids, gels, syrups, slurries, suspensions and the like, for oral or nasal ingestion by a patient to be treated.

Pharmaceutical compositions suitable for use in the present invention include compositions wherein the active ingredients are contained in an effective amount to achieve the intended purpose. For example, an effective amount of neutralizing may be that amount that protects against GVHD. Determination of effective amounts is well within the capability of those skilled in the art, especially in light of the disclosure provided herein.

In addition to the active ingredients, these pharmaceutical compositions may contain suitable pharmaceutically acceptable carriers comprising excipients and auxiliaries, which facilitate processing of the active compounds into preparations that can be used pharmaceutically. The preparations formulated for oral administration may be in the form of tablets, dragees, capsules, or solutions.

The pharmaceutical compositions of the present invention may be manufactured in a manner that is itself known (e.g., by means of conventional mixing, dissolving, granulating, dragee-making, levigating, emulsifying, encapsulating, entrapping or lyophilizing processes).

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are carbohydrate or protein fillers such as sugars, including lactose, sucrose, mannitol, or sorbitol; starch from corn, wheat, rice, potato, etc; cellulose such as methyl cellulose, hydroxypropylmethyl-cellulose, or sodium carboxymethylcellulose; and gums including arabic and tragacanth; and proteins such as gelatin and collagen. If desired, disintegrating or solubilizing agents may be added, such as the cross-linked polyvinyl pyrrolidone, agar, alginic acid or a salt thereof such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, (i.e., dosage).

Pharmaceutical preparations that can be used orally include push-fit capsules made of gelatin, as well as soft, sealed capsules made of gelatin and a coating such as glycerol or sorbitol. The push-fit capsules can contain the active ingredients mixed with a filler or binders such as lactose or starches, lubricants such as talc or magnesium stearate, and, optionally, stabilizers. In soft capsules, the active compounds may be dissolved or suspended in suitable liquids, such as fatty oils, liquid paraffin, or liquid polyethylene glycol with or without stabilizers.

Compositions comprising a compound of the invention formulated in a pharmaceutical acceptable carrier may be prepared, placed in an appropriate container, and labeled for treatment of an indicated condition.

The pharmaceutical composition may be provided as a salt and can be formed with many acids, including but not limited to hydrochloric, sulfuric, acetic, lactic, tartaric, malic, succinic, etc. Salts tend to be more soluble in aqueous or other protonic solvents that are the corresponding free base forms. In other cases, the preferred preparation may be a lyophilized powder in 1 mM-50 mM histidine, 0.1%-2% sucrose, 2%-7% mannitol at a pH range of 4.5 to 5.5 that is combined with buffer prior to use.

For any compound used in the method of the invention, the therapeutically effective dose can be estimated initially from cell culture assays. Then, preferably, dosage can be formulated in animal models (particularly murine models) to achieve a desirable circulating concentration range that adjusts neutralizing levels.

A therapeutically effective dose refers to that amount of neutralizing agent that ameliorates symptoms of the disease state. Toxicity and therapeutic efficacy of such compounds can be determined by standard pharmaceutical procedures in cell cultures, experimental animals or transgenic animals, e.g., for determining the LD₅₀ (the dose lethal to 50% of the population) and the ED₅₀ (the dose therapeutically effective in 50% of the population). The dose ratio between toxic and therapeutic effects is the therapeutic index, and it can be expressed as the ratio LD₅₀/ED₅₀. Compounds that exhibit large therapeutic indices are preferred. The data obtained from these cell culture assays and additional animal studies can be used in formulating a range of dosage for human use. The dosage of such compounds lies preferably within a range of circulating concentrations that include the ED₅₀ with little or no toxicity. The dosage varies within this range depending upon the dosage form employed, sensitivity of the patient, and the route of administration.

The exact dosage is chosen by the individual physician in view of the patient to be treated. Dosage and administration are adjusted to provide sufficient levels of the active moiety or to maintain the desired effect. Additional factors, which may be taken into account, include the severity of the disease state; age, weight, and gender of the patient; diet, time and frequency of administration, drug combination(s), reaction sensitivities, and tolerance/response to therapy. Long acting pharmaceutical compositions might be administered every 3 to 4 days, every week, or once every two weeks depending on half-life and clearance rate of the particular formulation.

### III. Other Research and Therapeutic Applications of the Present Invention

The present invention also finds utility in in vitro and ex vivo applications, as well as in vivo research studies. In one embodiment of the invention, the neutralization of GVHD promoting cytokines and/or their respective receptors is important in the elucidation of not only competing pathways, but also synergistic pathways of the way the cytokines function in their natural state. The present invention provides methods and agents for neutralizing particular cytokines, thereby providing a means for studying the role of other non-neutralized cytokines. In another embodiment, the neutralization of particular cytokines can be done in a dose-dependent manner thereby permitting study of the interaction of the various cytokines as well as the pharmacokinetics of the cytokine interaction. In still another embodiment, the role of non-neutralized cytokines may be elucidated with the neutralization of effects caused by particular cytokines that may mask the effects of the non-neutralized cytokines.

In still further embodiments, the agents and methods provided herein finds use in in vitro, ex vivo, or in vivo screening methods. The effectiveness of the agents described herein provides a baseline of effect known to prevent GVHD. An embodiment includes the use of the agents described herein to provide a baseline of comparison for new agents to determine their efficacy for treatment of GVHD. Further embodiments of this screening method include, but are not limited to, in vitro assays in an array such as multi-well plates. In other embodiments, the screening method comprises use of cells affected by the cytokines plated in an array wherein particular wells are treated with the neutralized cytokines and other wells are treated with the new test compounds. The effects on the cells are then compared to determine if the new test compounds would be efficacious in treating GVHD.

In still other embodiments, where inflammatory cytokines have been implicated in other forms of pulmonary injury, it is contemplated that the present invention can prophylactically protect against such injury and/or be used to treat such injury. For example, in a study of swine influenza virus, it was found that pigs had increased levels of IFN-α, TNF-α and IL-1. (Van Reeth and Nauwynck, Vet. Res. 31:187-213 (2000). The investigators suggested that proinflammatory cytokines may be important mediators of viral respiratory diseases in pigs. Therefore, it is contemplated that the present invention may have use in treating viral respiratory diseases in livestock, like pigs. It is also contemplated where lipopolysaccharide (LPS) promotes injury through increasing levels of cytokines, such as, but not limited to IFN-γ, TNF-α, and interleukins, the present invention is useful to either prevent or minimize such injury. Such treatment would not be limited to humans, but also to other animal species exposed to increased LPS.

### IV Experimentation Conducted During the Development of the Present Invention

### Enhanced GVHD Mortality and Morbidity in Aged Recipients of Allogeneic BMT

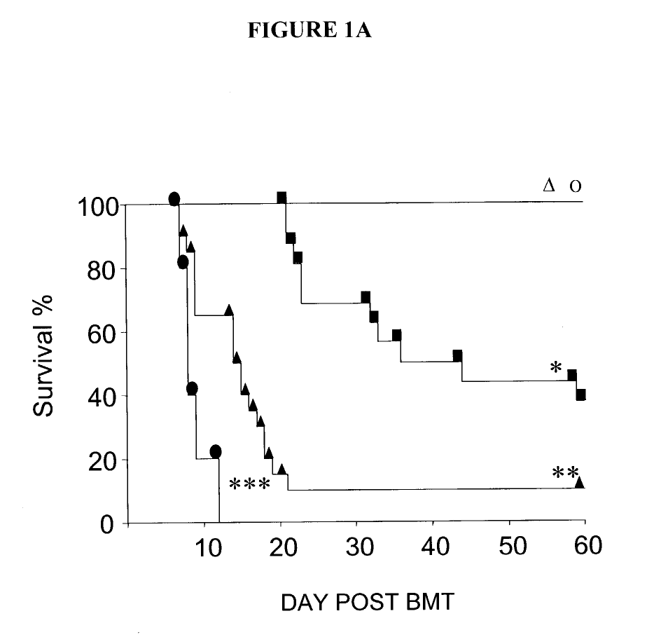

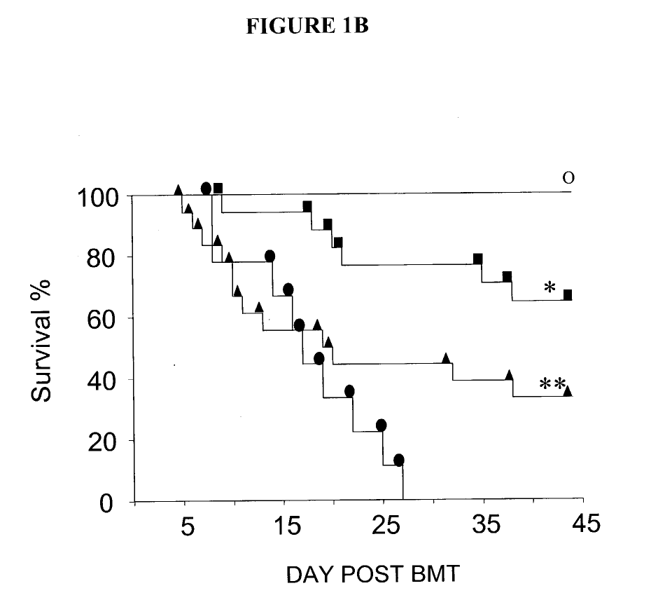

In an embodiment of the invention, the effect of recipient age on the severity of GVHD was shown in well-established murine models of GVHD elicited by both major and minor histocompatibility antigen differences between donors and recipients. Young (2 months old), old (14-16 months), and very old (22 months) B6D2F1 (H-2^{b/d}) mice were irradiated with 11 Gy TBI and transplanted with 5×10⁶ BM cells and 2×10⁶ splenic T cells from B6 (H-2^{b}) donors, as described in infra. As shown in FIG. 1A, recipient age is a continuous variable with respect to GVHD severity. Very old (filled circles, n=5), old (filled triangles, n=20), and young (filled squares, n=16) B6D2F1 mice underwent allogeneic BMT as described infra. Very old B6D2F1 (open circle, n=3) and old (open triangle, n=7) mice that received syngeneic BMT served as negative GVHD controls. Very old B6D2F1 recipients had significantly more rapid mortality than old recipients (***P=0.03), and old recipients died significantly faster than young recipients did (**P<0.005). Syngeneic control mice exhibited 100% survival during the observation period (circles and triangles). Severe GVHD developed in very old recipients, with 100% mortality by day 11. In old mice, mortality was slightly but significantly delayed (P=0.03), and it was further delayed in young recipients of allogeneic BMT, 50% of which were alive on day 50 (P<0.01). All old and very old recipients of syngeneic BMT survived the entire observation period, ruling out conditioning-related toxicity as a cause of death. In this particular embodiment, clinical GVHD scores were assessed by a standard scoring system that sums changes in five parameters (see infra). Scores were significantly greater in very old recipients compared with old recipients (7.2±0.1 versus 5.2±0.3, respectively, P<0.05). Clinical GVHD scores of old mice were also significantly higher compared with those of young mice (5.2±0.3 versus 4.3±0.1, respectively, P<0.01). Similar results were observed in a second allogeneic donor/recipient strain combination (B6→B6SJLF1) where old (16 months, n=9) and young (2 months, n=6) mice were used as recipients. Day 50 survival (55% in old mice versus 100% in young mice, P=0.016) and GVHD scores (3.7±0.5 in old mice versus 1.4±0.2 in young mice, P<0.01) demonstrated more severe GVHD in old mice at all timepoints.

In another embodiment, increased GVHD with age was also seen in a minor histocompatibility antigen-mismatched mouse model (B10.BR→CBA). See FIG. 1B. Very old (filled circles, n=9), old (filled triangles, n=17), and young (filled squares, n=17) CBA mice were transplanted from B10.BR donors in the same fashion described above. Very old CBA mice that received syngeneic BMT served as negative GVHD controls (open circle, n=7). Very old CBA mice had greater mortality than old mice had (P=0.2), and old mice died more quickly than young mice (**P=0.01). Syngeneic mice exhibited 100% survival during the observation period. Compared with young mice (2-3 months of age), old mice (12-14 months) died significantly more rapidly (P=0.01). All very old mice (22 months) developed more severe GVHD than both other groups and died within 26 days; age was a significant continuous variable with respect to GVHD, according to Cox regression analysis (P<0.001).

In further embodiments of the present invention, increased GVHD was also observed in old compared with young recipients after a variety of conditioning regimens, including cyclophosphamide alone. B6D2F1 mice were treated with 100 mg/kg cyclophosphamide on both day-2 and day-1 and transplanted with 5×10⁶ B6 BM cells and 2×10⁷ B6 spleen cells. Day 40 survival in 16-month-old BMT recipients was significantly decreased compared with that of young mice (0% versus 83%, P=0.01), and aged recipients displayed significantly more clinical acute GVHD than young recipients did (5.5±0.3 versus 3.2±0.2, respectively, P<0.01). Thus, increased GVHD mortality and morbidity in aged recipients was neither strain- nor conditioning-dependent.

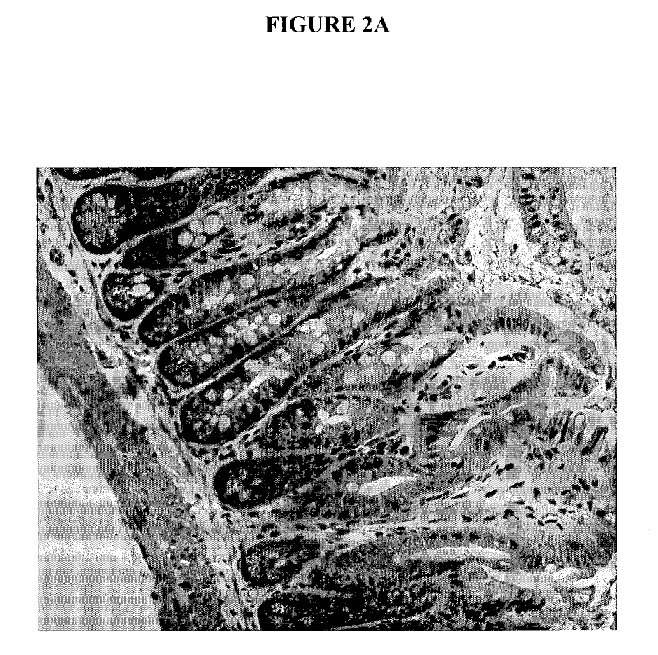

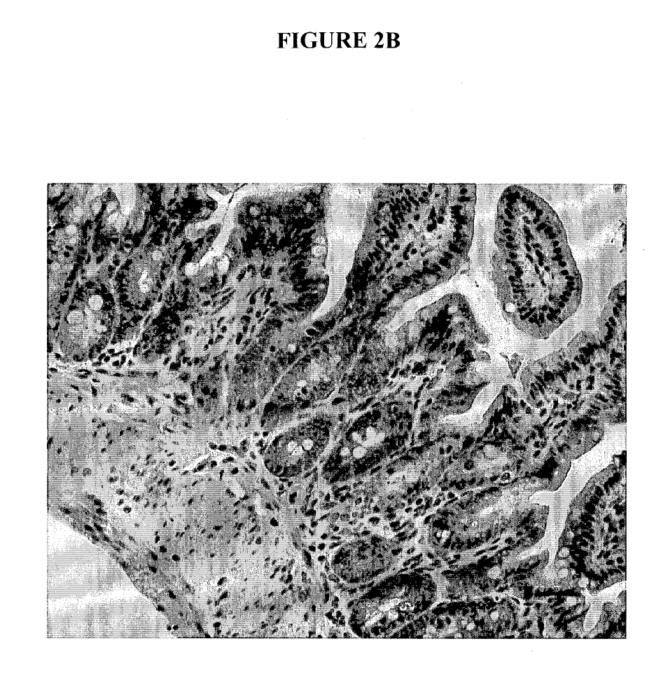

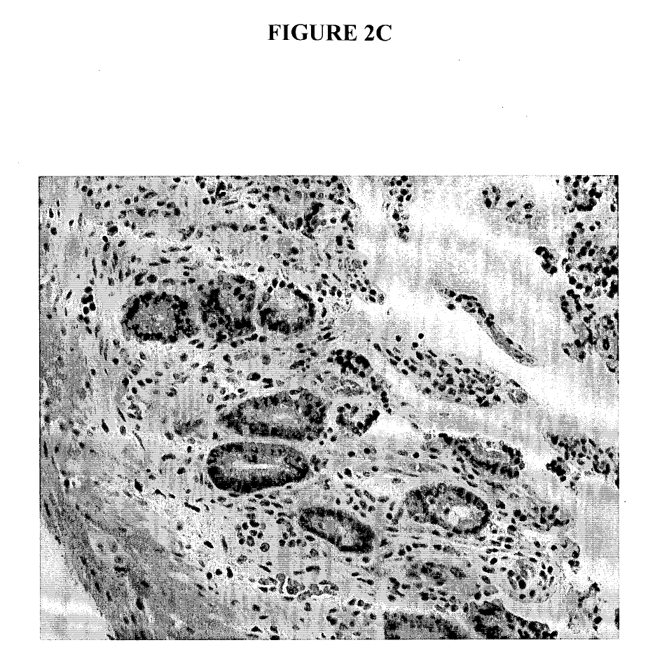

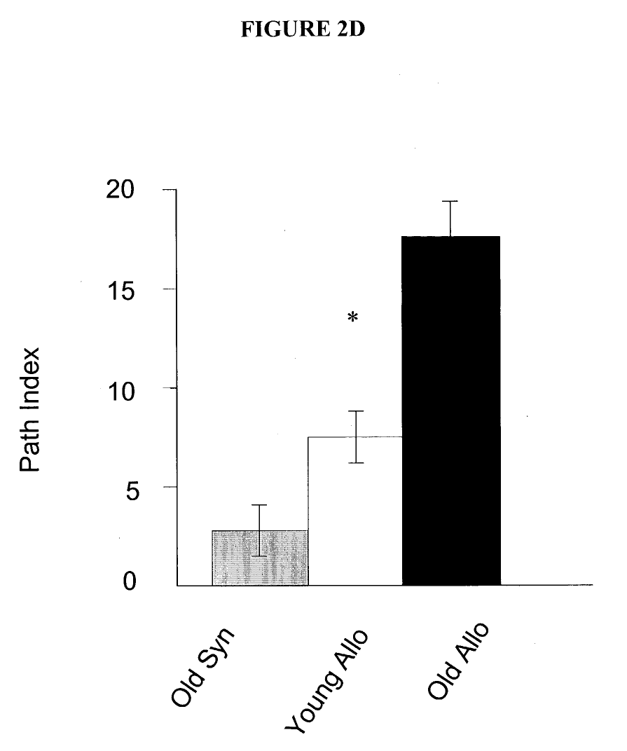

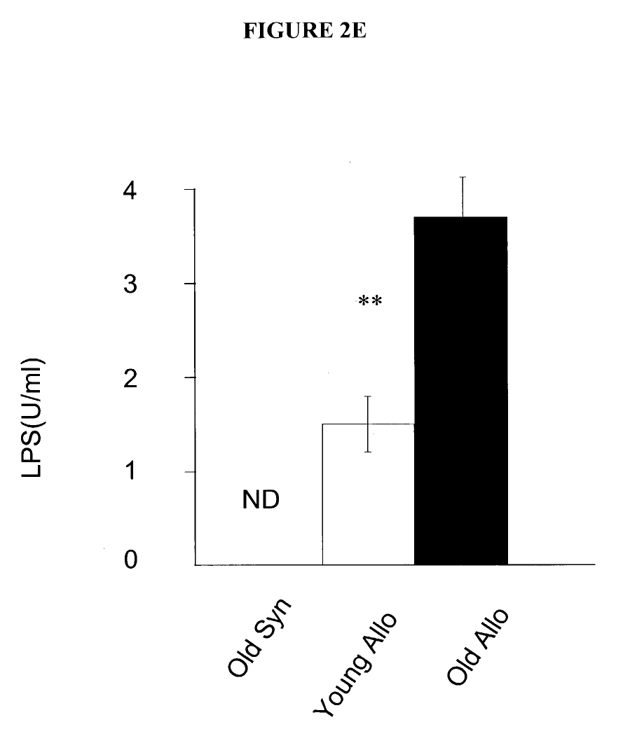

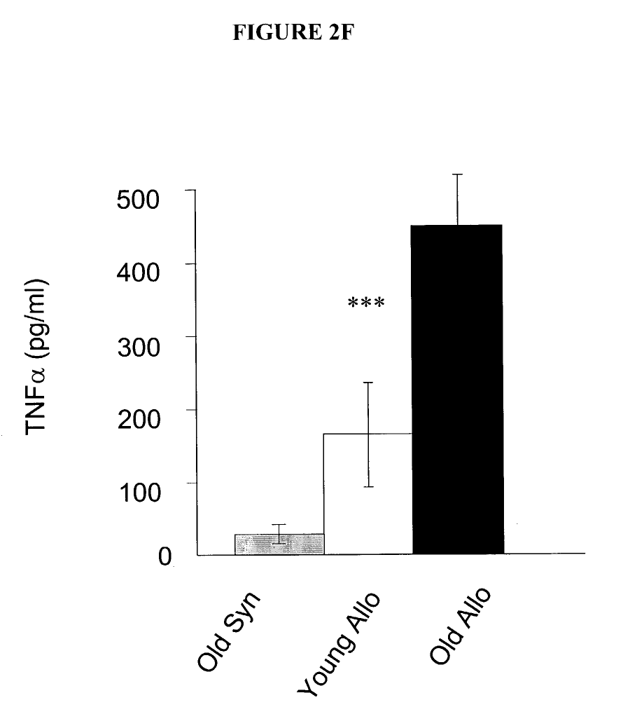

In another embodiment of the present invention, accelerated GVHD in old mice was shown by histopathologic analysis of the small and large intestine in the B6→B6D2F1 model. Acute GVHD histology is characterized by diffuse apoptosis, lymphocytic infiltrate, brush-border loss, mucosal sloughing into the lumen, epithelial degeneration, and crypt regeneration (see FIGS. 2A, B and C). Age exacerbates GI tract GVHD and increases serum LPS and TNF-α. B6D2F1 mice were transplanted as described supra. FIGS. 2A, B, and C show small bowel histology at day 7 in BMT recipients conditioned with 11 Gy TBI. Coded slides were evaluated for villus blunting, crypt destruction, loss of enterocyte brush border, luminal sloughing of cellular debris, and lamina propria lymphocytic infiltrate. Tissue damage of old syngeneic mice was minimal (see FIG. 2A). The small bowel of old allogeneic mice (see FIG. 2C) exhibited more severe villus blunting, crypt destruction changes, crypt atrophy, and increased lymphocytic infiltrate than did that of young allogeneic mice (see FIG. 2B). Original magnification was ×200.

Histologic scores of individual criteria were summed to produce a semiquantitative pathology index as previously described (Hill and Ferrara, Blood, 95:2754-2759 (2000)). FIG. 2D shows coded slides from each group (old syngeneic (gray bar), young allogeneic (white bar), and old allogeneic mice (black bar)) were scored for each parameter on a scale of 0-4 and summed (n=4 in each group). In a comparison of young versus old allogeneic mice, a significant difference is seen with a *P<0.03. Damage to the GI tract was twice as severe in old recipients as in young allogeneic recipients (see FIG. 2D). Mice were transplanted as described infra at Example 1 and serum was obtained by performing heart puncture on day 7 after BMT. LPS (see FIG. 2E) and TNF-α (see FIG. 2F) data represent the mean ±SE of 9 young (white bar) and 9 old (black bar) animals with p-values of **P<0.01 and ***P=0.03 in young versus old allogeneic mice. Serum levels of LPS and TNF-α were also significantly increased in older recipients, consistent with the pathologic data (see FIGS. 2E and 2F). Data were combined from animals transplanted in two separate experiments. Thus by all clinical, pathologic, and biochemical indices, acute GVHD was worse in old allogeneic recipients than in young allogeneic recipients in this particular embodiment.

### Aged Host Natural Killer Cells do not Cause Increased GVHD in Aged Recipients

Host natural killer (NK) cells are known to decline with age in several mouse strains, and NK cells play an important role in hybrid resistance in the B6→B6D2F1 BMT model (Albright and Albright, Proc. Natl. Acad. Sci. USA, 80:6371-6375 (1983); Mikael et al., Growth Dev. Aging, 58:3-12 (1994). In an embodiment of the invention, NK cells of BMT recipients were depleted according to a published protocol (Bix et al., Nature, 349:329-231 (1991). This excluded the possibility that a decline of NK cell function with age was responsible for the accelerated acute GVHD seen in old mice. Mice were treated with 200 μg of anti-NK1.1 mAbs on days-2 and -1, which depleted NK1.1⁺ cells as measured by FACS (data not shown). At day 0, mice were irradiated with 11 Gy TBI and transplanted as described infra. GVHD mortality in old NK-depleted recipients was significantly higher than in young NK-depleted mice (median survival time, 10 days versus 21 days, respectively; P=0.01). Clinical GVHD also remained more severe in old mice after NK cell depletion (6.2±0.3 versus 4.6±0.3 in young mice, P<0.01). Depletion of NK cells from BMT recipients, thus, had no impact on the effects of age on GVHD severity.

### Enhanced Donor T Cell Responses to Host Antigens in Aged Allogeneic BMT

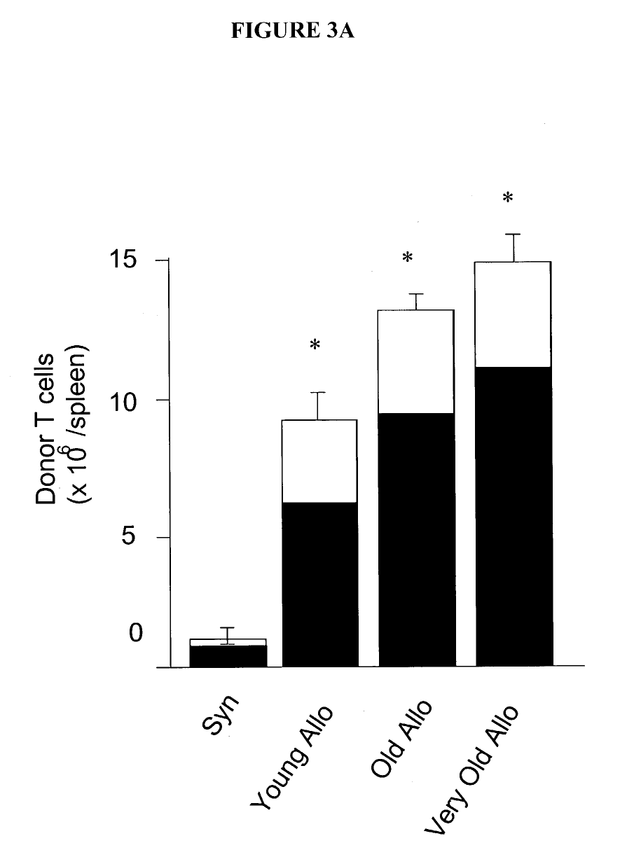

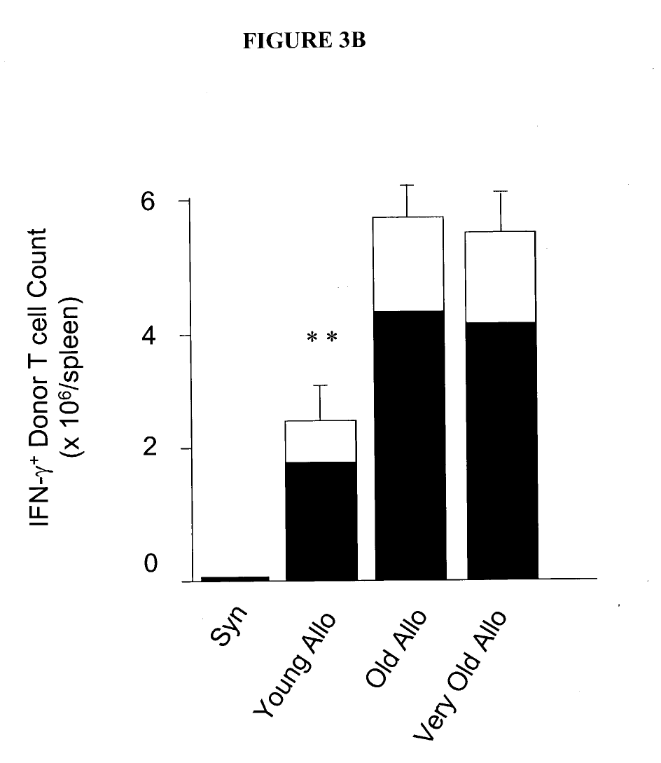

Acute GVHD requires activation of donor T cells by host APCs (Shlomchik et al., Science, 285:412-415 (1999)). In an embodiment of the present invention, donor T cell expansion in the spleens of BMT recipients 4 days after transplant were analyzed. Using B6.Ly5.2 congenic donors, significantly increased numbers of donor T cells (CD4⁺, CD8⁺, CD45.1⁺) that correlated with advancing recipient age were observed. This increase was mainly due to the increase of donor CD4⁺ T cells and not CD8⁺ T cells (see FIG. 3A). Mice of different ages were transplanted with 5×10⁶ T cells after radiation with 11 Gy. Splenocytes were harvested on day 4 (n=3/group). Donor T cell phenotype (CD4⁺, CD8⁺, and CD45.1⁺) was determined by FACS analysis as described in infra. Data represent the mean +SE of CD4⁺ (black bar) and CD8⁺ cells (white bar). Age was a significant continuous variable with respect to CD4⁺ donor T cell expansion according to Cox regression analysis (*P=0.02). Data from one of three similar experiments is shown. FACS analysis of donor cells by annexin staining showed no difference in the percentage of apoptotic donor cells in young versus old recipients, ruling out less activated induced cell death in old recipients as a cause for the increased T cell expansion in aged recipients (Reddy et al., J. Exp. Med. 194:1433-1440 (2001)).

In another embodiment, flow automated cytometric analysis of intracellular IFN-γ, an important cytokine mediator of acute GVHD, showed a twofold increase in the number of IFN-γ-expressing CD4⁺ donor T cells in the spleens of old recipients. The number of IFN-γ-producing donor T cells was very similar in old and very old allogeneic recipients compared with young allogeneic recipients (see FIG. 3B). IFN-γ production from donor cells was determined by intracytoplasmic staining of IFN-γ after cells were purified by Ficoll density-gradient centrifugation and incubated for 4 hours with PMA, ionomycin, and brefeldin A. **P<0.002, young versus old and very old. One of three similar experiments is shown.

### CD4⁺ Donor Cells are Responsible for Increased Acute GVHD in Old Mice

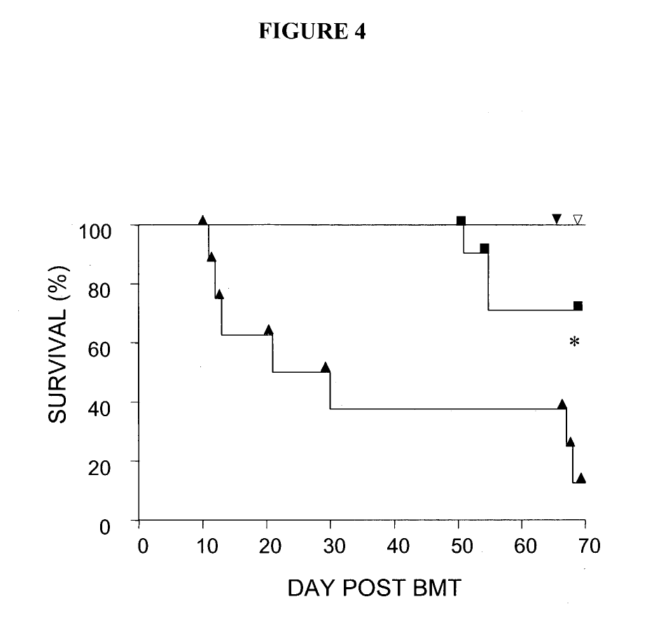

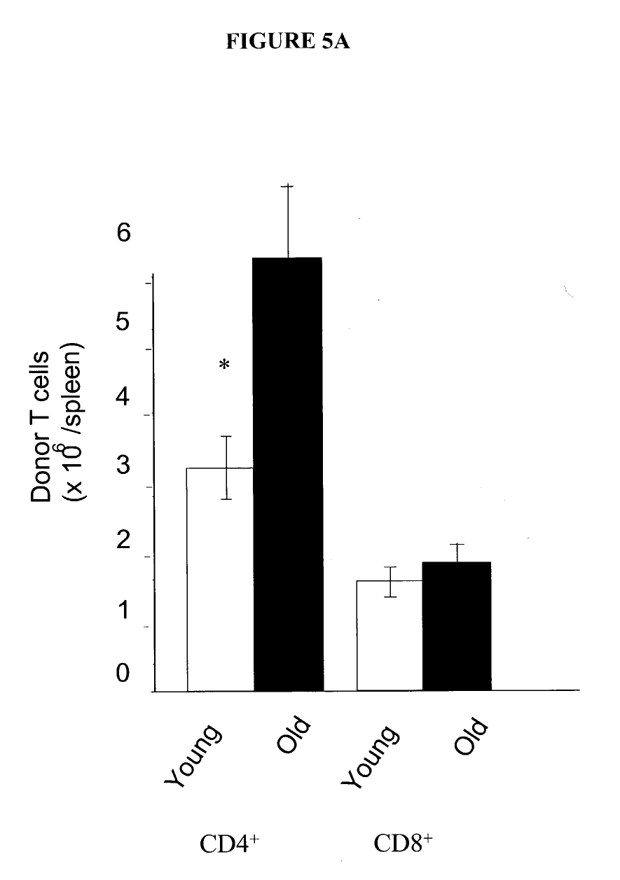

In an embodiment of the present invention, the role of donor CD4⁺ T cells in severity of GVHD in old recipients was evaluated. Either CD4⁺ or CD8⁺ T cells from donor B6 splenic T cells were depleted with mAb coupled to magnetic beads using the AutoMACS system as described infra. FACS analysis showed that the purity of the resulting donor T cells was always greater than 95%. Young and old B6D2F1 recipients were transplanted with 5×10⁶ T cell depleted (TCD) BM cells together with 1×10⁶ CD4⁺ or CD8⁺ T cells. Depletion of CD4+T cells from the donor-cell inoculum effectively eliminated GVHD mortality, and both young and old recipients of CD8⁺ cells survived the entire observation period (see FIG. 4). CD4⁺ donor T cells appear to induce GVHD mortality in old allogeneic recipients. Old (triangles, n=8) and young (squares, n=10) B6D2F1 mice received 11 Gy TBI and were injected with 5×10⁶ TCD BM and 1×10⁶ CD4⁺ splenic T cells from allogeneic B6 mice. In a comparison of old versus young mice, a significant difference was seen with a *P<0.005. Filled inverted triangle and open inverted triangle represent old (n=8) B6D2F1 and young (n=10) B6D2F1 mice, respectively, receiving 5×10⁶ TCD BM cells and 1×10⁶ CD8⁺ donor T cells. By contrast, old recipients of CD4⁺ T cells experienced significantly greater mortality than did young recipients (P<0.001). In this particular embodiment, CD4⁺ donor T cells are therefore the principal effectors in acute GVHD severity in this parent→F1 mouse model and are responsible for the increased GVHD in old recipients.

### Increased T Cell Stimulation by APCs from Aged Mice In Vivo and In Vitro

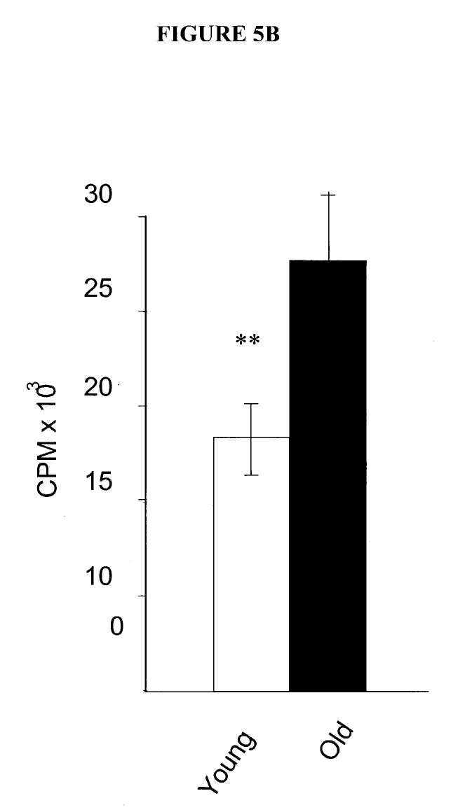

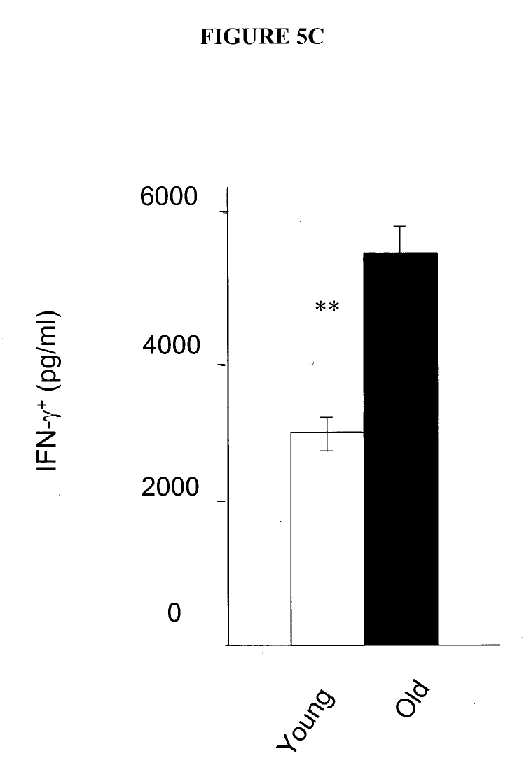

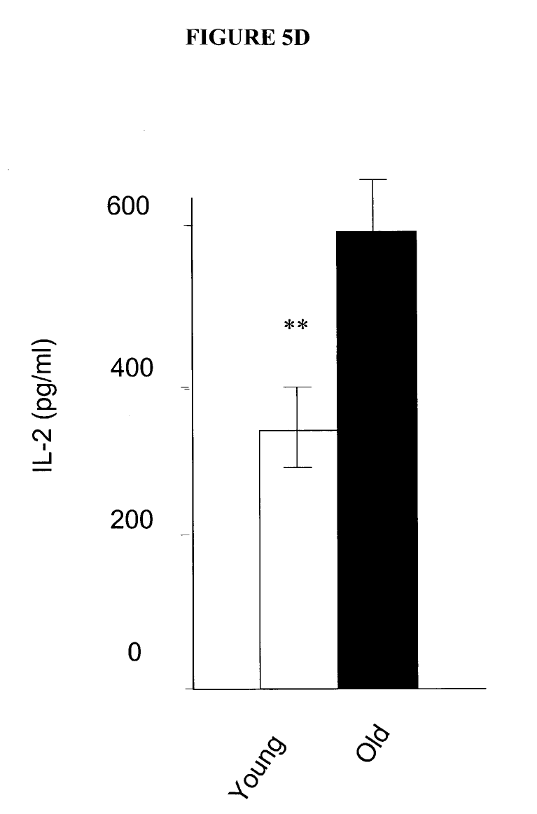

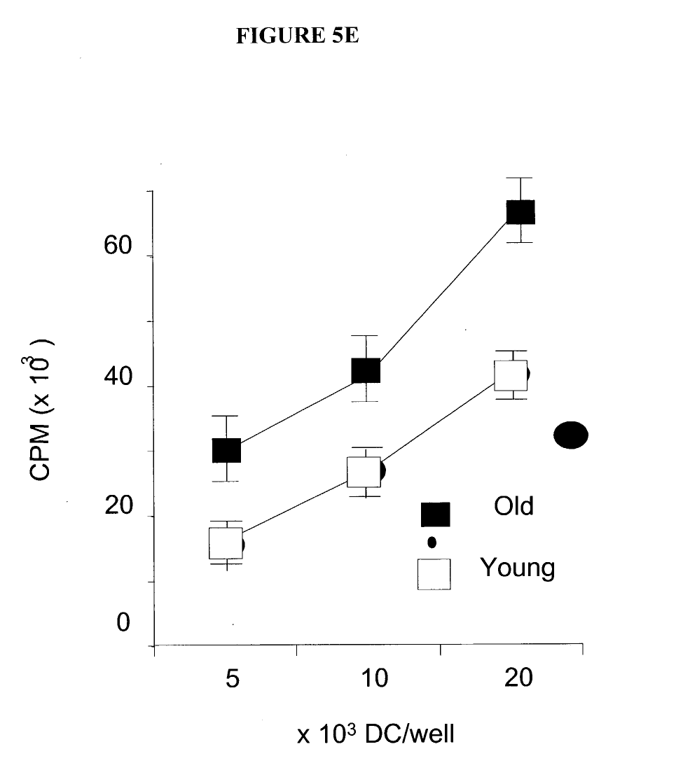

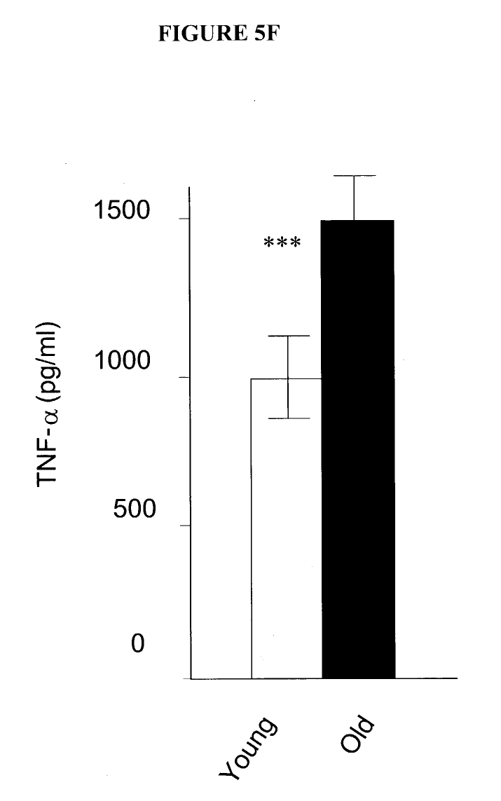

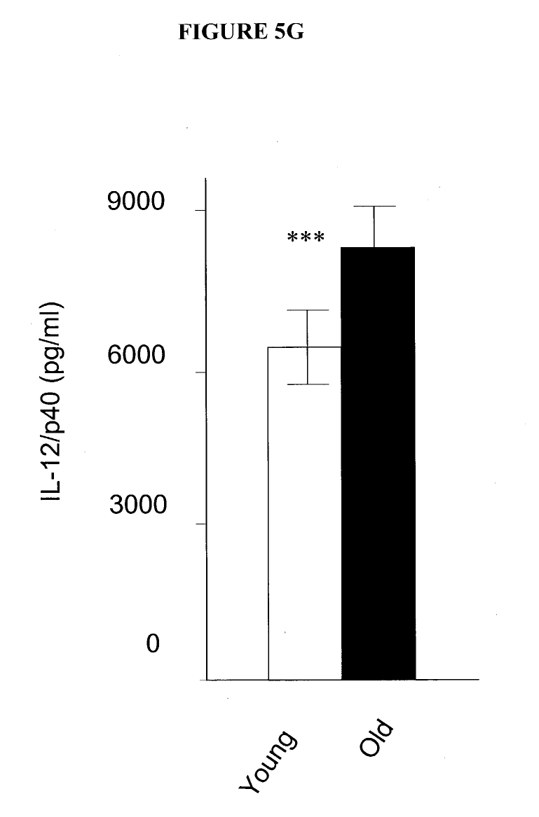

To investigate whether enhanced donor T cell responses in old recipients were secondary to increased susceptibility of the GI tract to TBI conditioning that would allow translocation of inflammatory stimuli to the systemic circulation, donor T cell expansion was analyzed in this embodiment of the invention. Nonirradiated young and old B6D2F1 mice were injected with 5×10⁷ splenic lymphocytes from B6.Ly5.2 donors for expansion studies. To exclude the impact of NK cells in this nonirradiated model, NK cells were depleted with anti-NK1.1 mAb as described infra. Spleens were harvested on day 4. Donor T cell phenotype (CD4⁺, CD8⁺, and CD45.1⁺) was determined by FACS analysis as described infra. Donor T cell expansion was twofold greater in the spleens of nonirradiated old recipients, due primarily to expansion of CD4⁺ T cells (see FIG. 5A).

In another embodiment, the ability of APCs from freshly isolated B6D2F1 splenocytes to stimulate naive B6 responders in vitro was also assessed. Young splenic T cells from B6 mice were cultured with irradiated spleen cells from either young B6D2F1 or old B6D2F1 animals. Proliferation and IL-2 and IFN-γ production were measured as described infra. As shown in FIGS. 5B, C, and D, B6 T cells proliferated more rapidly and produced greater amounts of IFN-γ and IL-2 in response to B6D2F1 stimulator cells from old animals than from young animals. APCs from very old mice also showed greater stimulation of allogeneic responses than did APCs from old mice. Proliferation in B10.BR anti-CBA mixed lymphocyte reaction (MLR) (which is a CD8⁺ T cell-dependent minor antigen H mismatched system) (Korngold and Sprent. T cell subsets in graft-versus-host disease in Graft-versus-host disease: immunology, pathophysiology, and treatment. Marcel Dekker Inc. New York, N.Y., USA. pp. 31-50 (1990)) was also increased with old stimulators (4,732±370 versus 8,735±1,010 cpm, P<0.001). FACS analysis of the spleen cells did not show increases in the numbers of CD11b⁺ macrophages or CD11c⁺ dendritic cells (DCs) with increasing age.

Since dendritic cells are the most potent APCs (Steinman, Annu. Rev. Immunol., 9:271-296 (1991)), the allostimulatory capacity of BM-derived DCs from young versus old mice were compared in this particular embodiment. DCs were generated in a standard fashion as described infra. BM-derived DCs were irradiated and cultured with 1×105 T cells from B6 mice. DCs from old mice induced greater proliferation of allogeneic T cells than did DCs from young mice (see FIG. 5E). T cell proliferation was determined by (³H) thymidine incorporation. Significant differences in allogeneic T cell responses to old DCs (filled squares) versus young DCs (open squares) were see with a P<0.01.

In a further embodiment, FACS analysis of dendritic cells showed that costimulatory molecules, including CD80, CD86, and CD40, were not consistently increased, but MCH II expression of DCs was consistently up-regulated with increasing age (mean fluorescence intensity: 12 versus 29 versus 41 units). TNF-α and IL-12, known to play important roles in augmenting alloreactive T cell response, were also produced in significantly greater amounts by old DCs than by young DCs after LPS stimulation (see FIGS. 5F and G). TNF-α and IL-12 p40 secretion from. DCs were stimulated with 0.1 μg LPS/ml for 4 hours (TNF-α) or 24 hours (IL-12 p40). Each graph represents one of three similar experiments. *P=0.03, **P<0.01. All mice tested negative for infectious pathogens, ruling out increased viral burden and expression of neoantigens as the cause for increased allostimulation by old APCs.

### Aged APCs are Sufficient to Increase GVHD Severity

While an understanding of the mechanism is not required to practice the present invention and the present invention is not limited to any particular mechanism, the question of whether aged APCs were sufficient to induce more severe GVHD was investigated. In this particular embodiment, BM chimeras comprised of hematopoietic cells from old mice and nonhematopoietic cells (including all epithelial GVHD targets) from young mice. Young B6D2F1 mice (2 months of age) were irradiated with 11 Gy, followed by injection of 5×10⁶ BM cells from either young (2 months) or old (18 months) syngeneic B6D2F1 mice. This BMT procedure produced 100% donor engraftment by week 8 after transplantation. Four months later, old→young and young→young chimeric mice were used as recipients of allogeneic BMT. If increased age of APCs was the primary cause of increased GVHD severity, then old→young chimeras injected with old BM would have old APCs and should develop more intense disease than young→young chimeras, despite the identical age of the GVHD target tissues.

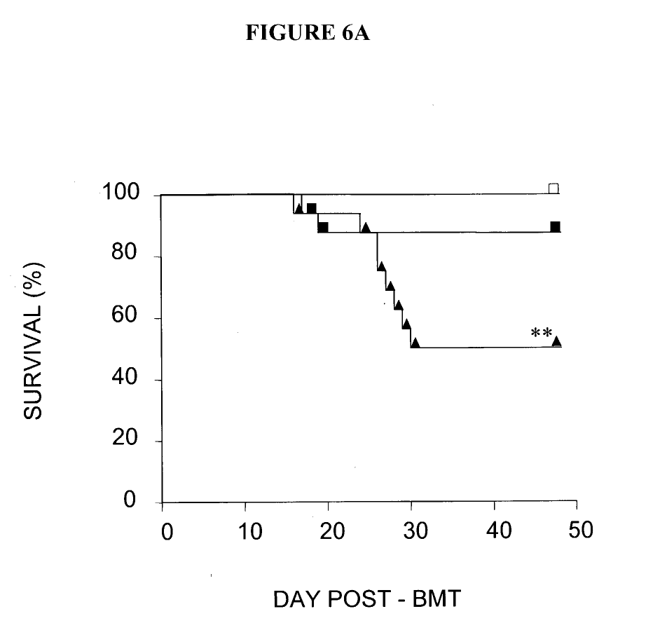

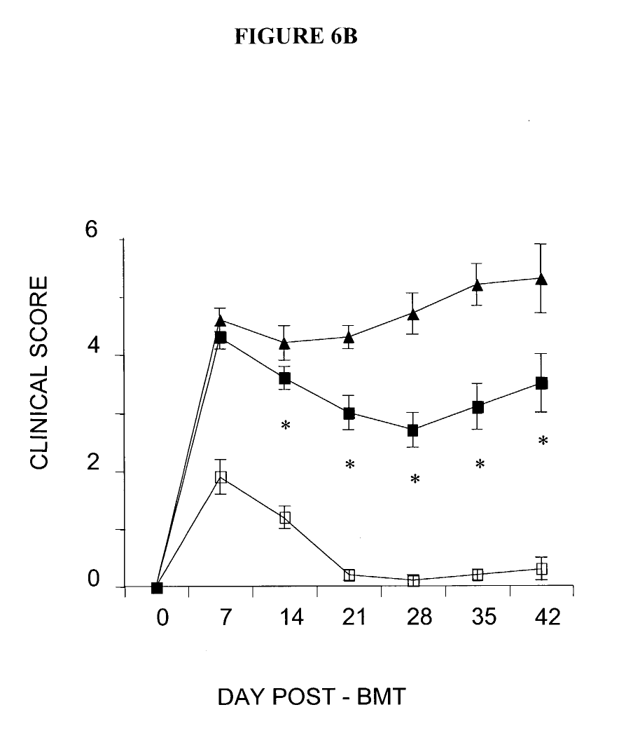

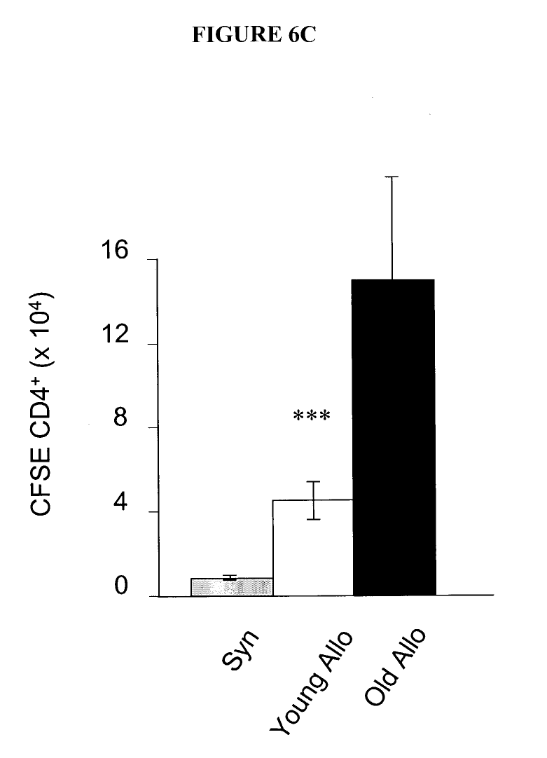

In this particular embodiment, the chimeric mice were transplanted with 2×10⁶ splenic T cells and 5×10⁶ BM cells from B6 donors after 7.5 Gy TBI. More specifically, young B6D2F1 mice first received 11 Gy TBI and were injected with 5×0⁶ BM cells from young (2 months old) or old (18 months) B6D2F1 mice. After allowing 3 months for hematopoietic engraftment and APC repopulation, chimeric mice were irradiated again with 7.5 Gy and injected with 5×10⁶ B6 BM cells and 2×10⁶ splenic T cells from B6 mice. The lower dose of 7.5 Gy was chosen to minimize conditioning toxicity while remaining myeloablative. As shown in FIG. 6A, old→young chimeric mice (triangles, n=16) had significantly more rapid mortality than did young→young chimeras (squares, n=16). **P<0.003 by log rank test. The old→young chimeras developed significantly more severe GVHD than did young→young chimeras, as measured by survival and clinical score. FIG. 6B shows the clinical GVHD score that was measured for this particular embodiment as described in infra. There was a significant difference between old donors versus young donors with a *P<0.05. GVHD scores are shown as mean ±SE. Although there was almost no mortality in young→young chimeras receiving young APCs (probably due to the decreased TBI conditioning), GVHD was present, as evidenced by the significantly increased clinical scores. Flow cytometric analysis confirmed 100% donor engraftment in both groups after the second BMT, ruling out mixed chimerism as a reason for reduced GVHD in young→young chimeras. Analysis of CFSE-labeled donor T cell response in vivo at day 3 after BMT confirmed greater CD4⁺ donor T cell expansion (see FIG. 6C). B6 donor T cells were labeled with CFSE as described infra and injected into chimeric recipients after 7.5 Gy TBI. Splenocytes were harvested and pooled (n=3/group) on day 3 after BMT and labeled with anti-CD4 as described in Methods. ***P<0.02. The percentage of cells undergoing at least two cell divisions in the old→young chimeras was higher than in the young→young chimeras (74% versus 65%), indicating increased T cell response to old APCs in vivo.

In vitro naive B6 T cell responders proliferated more rapidly in response to freshly isolated splenocytes from old chimera B6D2F1 mice than from young chimeric mice in MLR (29,853±2,066 versus 18,363±1,614, P<0.001). While an understanding of the mechanism is not required to practice the present invention and the present invention is not limited to any particular mechanism, these data indicate that old APCs are sufficient to induce an increased response from allogeneic donor T cells and cause more severe GVHD, irrespective of the age of the recipient target organs.

### MHC II^{-/-} Mice are Resistant to CD4-Mediated Acute GVHD

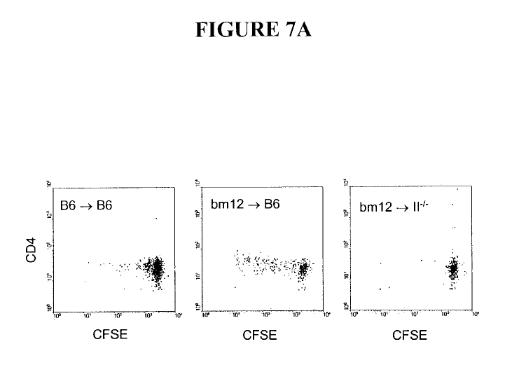

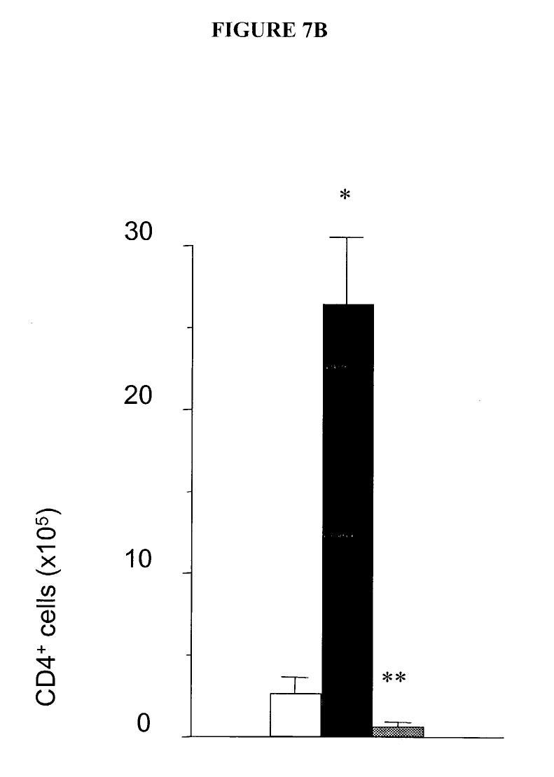

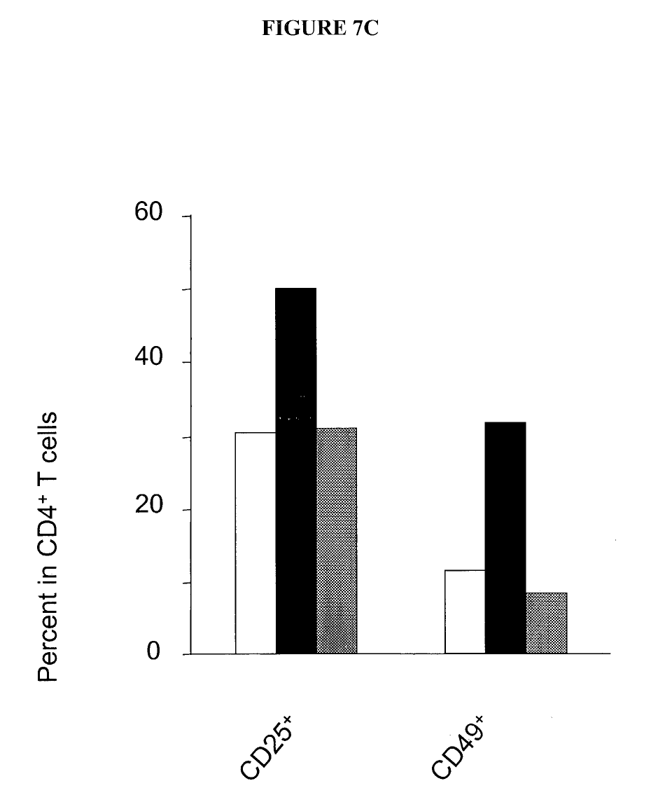

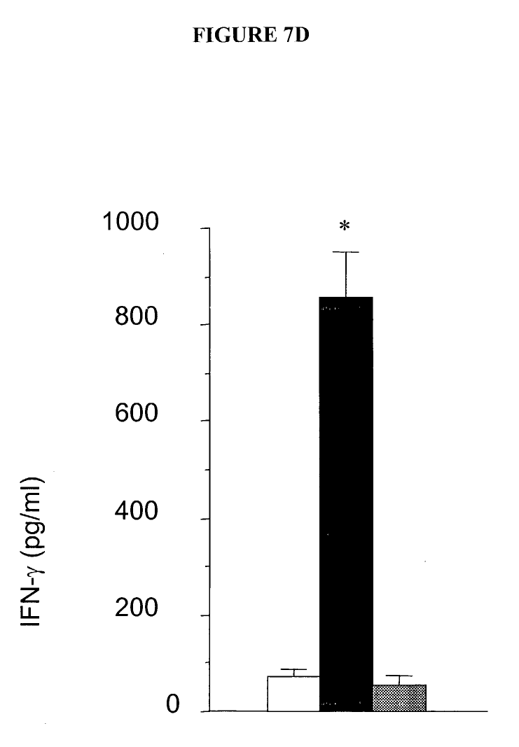

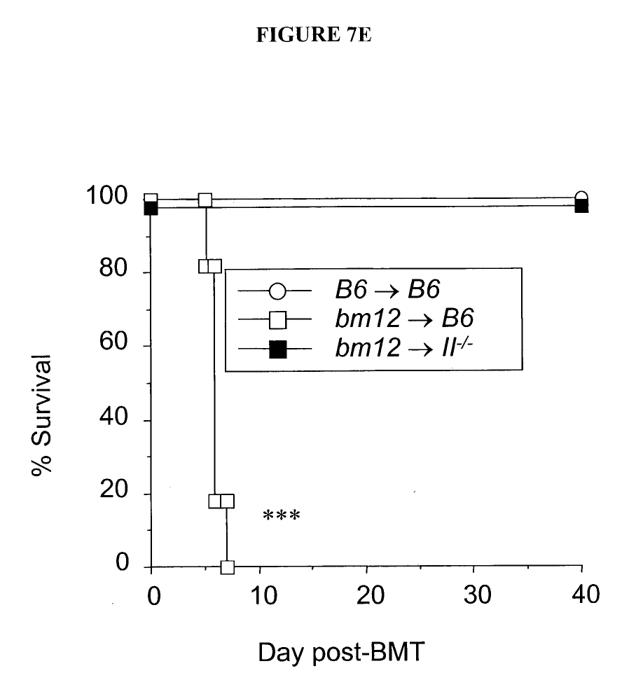

In an embodiment of the present invention, the requirement for the expression of MHC class II antigens to stimulate allogeneic CD4⁺ T cells was examined in vivo. Carboxy-fluorescein diacetate succinimidyl ester (CFSE)-labeled T cells from bm12 donors (see infra at Example 1) were transferred to B6 mice. The B6 mice had received 13 Gy total body irradiation (TBI) prior to transfer and were different from donor bm12 mice at a single MHC class II allele (Korngold and Sprent. T cell subsets in graft-versus-host disease in Graft-versus-host disease: immunology, pathophysiology, and treatment. Marcel Dekker Inc. New York, N.Y., USA. pp. 31-50 (1990)). FIG. 7A shows flow automated cytometric analysis of spleens 3 days after transfer showed that 42% of CFSE-labeled T cells from bm12 donor mice had progressed through at least one cell division in allogeneic wild-type (wt) B6 recipients (bm12→B6), whereas only 1% did so in allogeneic II^{-/-} B6 recipients (bm12→II^{-/-}). Cell division determined by FITC-CFSE is shown for CD4⁺ cells. 16% of donor T cells underwent “homeostatic” divisions after syngeneic transfer (B6→B6). This lymphopenia-driven homeostatic proliferation of CD4⁺ T cells requires TCR and MHC-peptides interactions (Paris et al., Science, 293: 293-297 (2001)).

In a further embodiment, six days after transfer of donor T cells, splenocytes were analyzed by flow cytometry for cell expansion and serum was collected (n=3/group). This lymphopenia-driven homeostatic proliferation was not seen in II^{-/-} mice, which resulted in lower CD4⁺ cell numbers in II^{-/-} recipients than in syngeneic controls (see FIG. 7B, B6→B6 (white bar), bm12→B6 (black bar), bm12→II^{-/-} (grey bar)). bm12 donor T cell expansion was confirmed in B6.Ly-5a recipients (CD45.1⁺) where >95% of splenic CD4⁺ T cells were of donor (CD45.2⁺) origin (data not shown). Expansion of bm12 CD4⁺ T cells in wt B6 recipients was associated with an increased expression of CD25 and CD49 (FIG. 7C) and an increased serum level of interferon (IFN)-γ (see FIG. 7D), findings which were absent in II^{-/-} recipients. Clinical GVHD scores in II^{-/-} recipients were also equivalent to syngeneic controls (data not shown). Data represent mean ±SD. *P<0.05 (B6→B6 vs. bm12→B6), **P<0.05 (bm12→II^{-/-} vs bm12→B6). When II^{-/-} or B6 mice were given 13 Gy TBI and then transplanted with BM and T cells from bm12 donor mice, wt B6 recipients experienced 100% mortality from GVHD by day 7, whereas 0% mortality was observed in II^{-/-} B6 recipients (see FIG. 7E, B6→B6 (∘, n=11), bm12→B6 (□, n=7), and bm12→II^{-/-} (▪, n=10) with a ***P<0.001 (□ vs. ▪)).

### MHC Class II Expression on Host APCs Alone is Sufficient to Stimulate Allogeneic CD4⁺ T Cells In Vivo

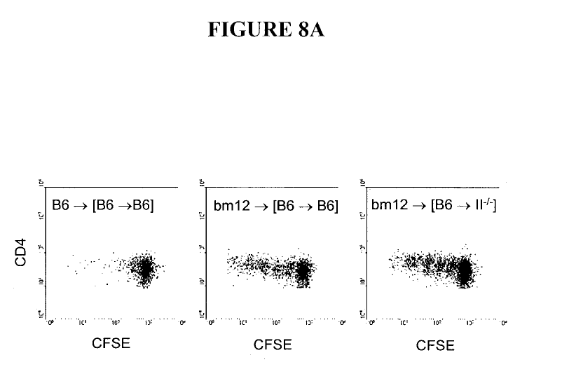

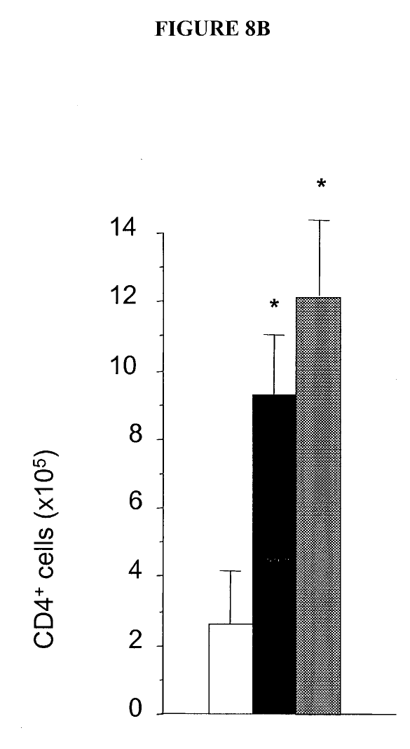

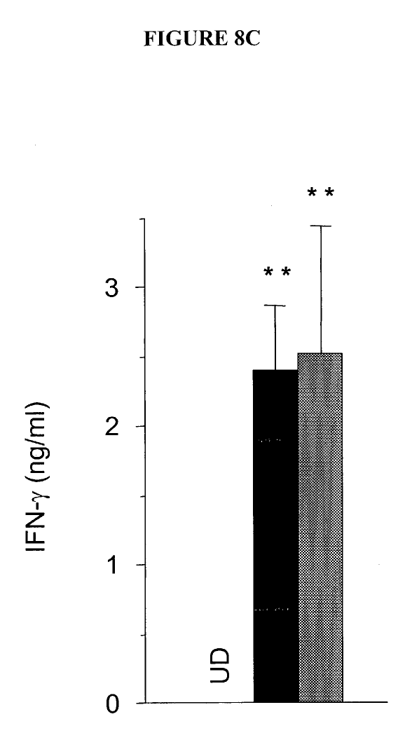

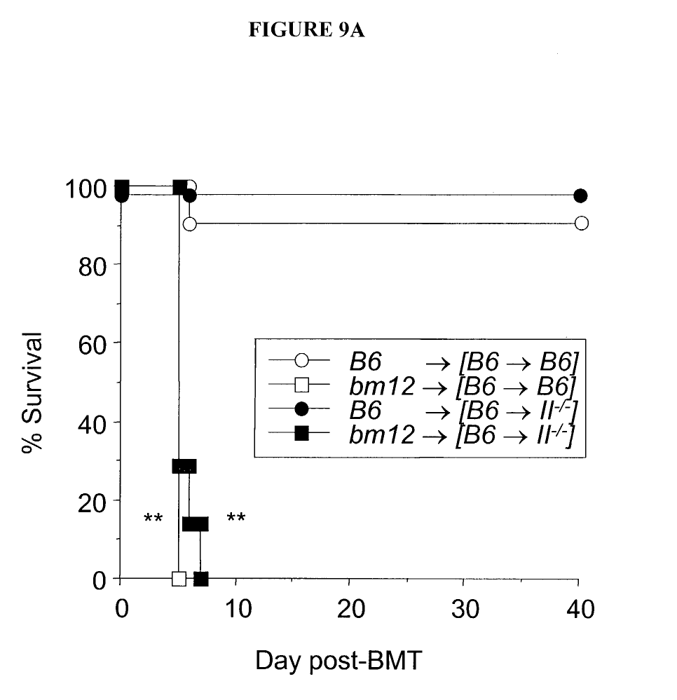

While an understanding of the mechanism is not required to practice the present invention and the present invention is not limited to any particular mechanism, the requirement for the expression of MHC class II on host APCs in this disease process was tested. In an embodiment of the invention, BM chimeras were generated to express MHC class II on BM-derived APCs, but lacking MHC class II on target cells by reconstituting lethally irradiated II^{-/-} mice with B6 BM cells ([B6→II^{-/-}] chimeras). Four months later, chimeras were re-irradiated and injected with CFSE-labeled splenic T cells from either bm12 or B6 donor mice. Splenocytes harvested 3 days after transfer were combined (n=3/group) and analyzed. Cell division determined by FITC-CFSE is shown for CD4⁺ cells. Flow automated cytometric analysis of splenic DCs isolated from these animals showed complete replacement of splenic DCs by donor BM cells with MHC class II expression on >95% of CD11c⁺ DCs (data not shown) as described (Krasinskas et al., Transplantation, 70:514-521 (2000)). Identically treated [B6→B6] chimeras were created as controls. Chimeras were reirradiated with 13 Gy TBI and injected with 4×10⁶ CFSE-labeled T cells from bm12 donors. Analysis of the spleen three days later showed equally robust cell division and significant proliferation of CD4⁺ bm12 donor T cells in both [B6→II^{-/-}] and [B6→B6] chimeric recipients compared to syngeneic B6→[B6→B6] controls (FIGS. 8A and B). No CD8⁺ T cell expansion was observed, confirming the stimulation of only CD4⁺ donor T cells in this system (data not shown). Serum levels of INF-γ at day 5 were markedly elevated in both [B6→II^{-/-}] and [B6→B6] chimera recipients compared to syngeneic controls (FIG. 8C, B6→[B6→B6] (white bar), bm12→[B6→B6] (black bar), bm12→[B6→II^{-/-}] (grey bar); UD=undetectable. Alloantigens on host APCs alone are therefore sufficient to induce CD4⁺ T cell expansion and cytokine secretion. Data represent mean ±SD. *P<0.05, **P<0.01 (bm12→[B6→B6] vs. B6→[B6→B6], bm12→[B6→II^{-/-}] vs. B6→[B6→B6]).

### Acute GVHD Does Not Require MHC Class II Alloantigen Expression on Host Epithelium

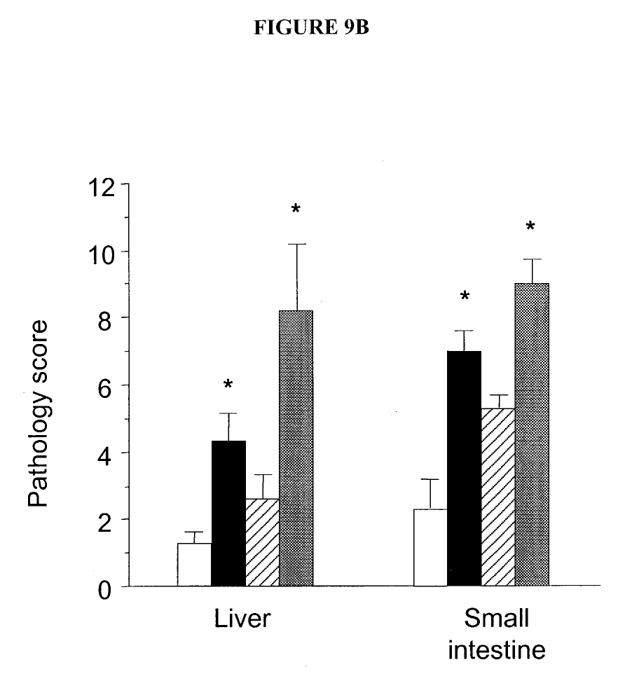

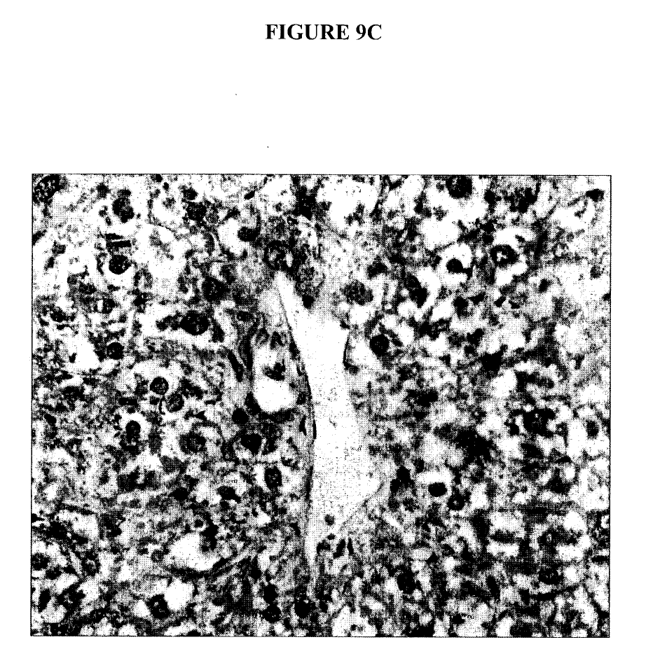

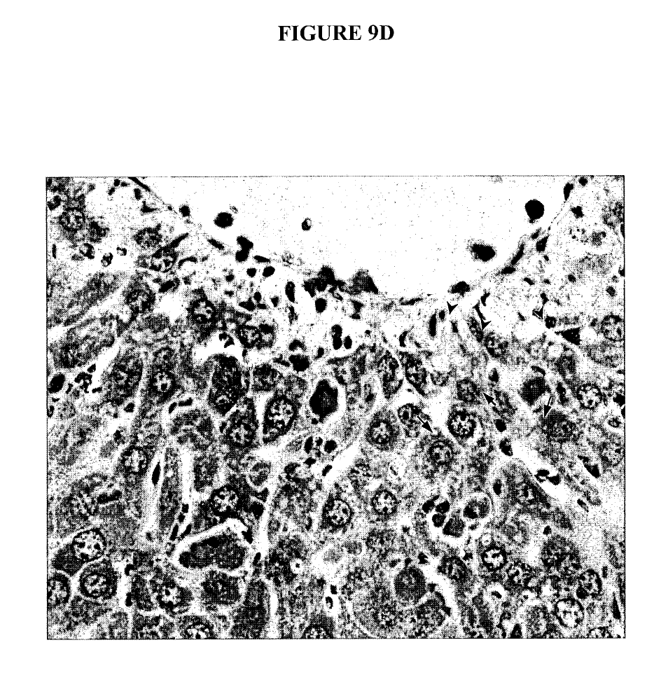

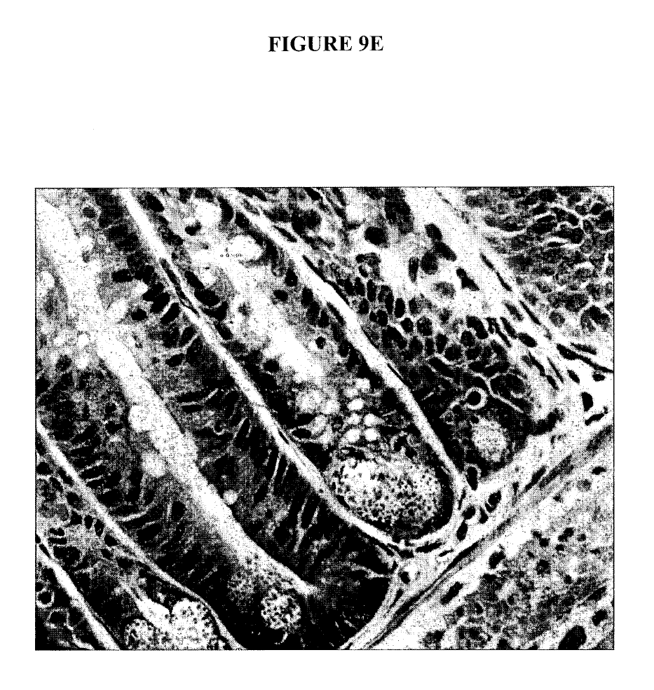

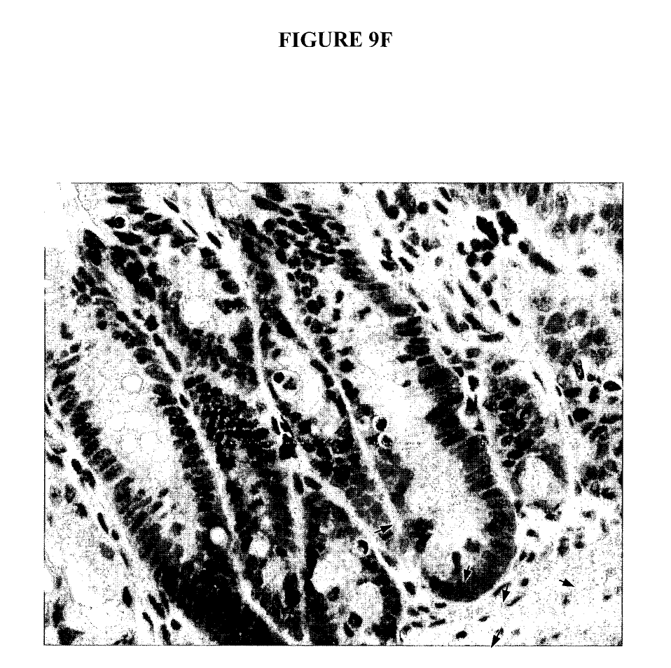

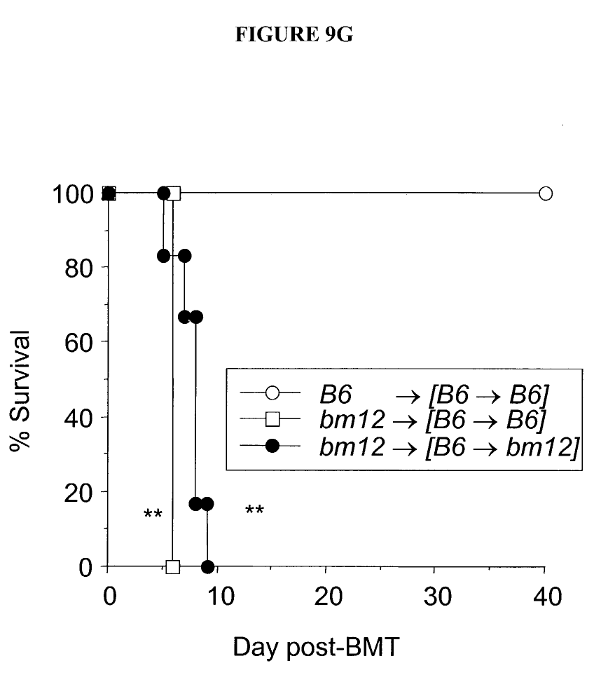

While an understanding of the mechanism is not required to practice the present invention and the present invention is not limited to any particular mechanism, donor CD4⁺ T cell activation were tested for induction of GVHD in the absence of MHC class II on nonhematopoietic target cells. In an embodiment of the present invention, [B6→II^{-/-}] and [B6→B6] chimeras were created and four months later were transplanted with BM and T cells from bm12 donors after 13 Gy TBI. As expected, GVHD was very severe in [B6→B6] recipients of bm12 T donors: all recipients died at day 5 of BMT in contrast to 90% survival of recipients of syngeneic B6 donors (FIG. 9A, B6→[B6→B6] (∘, n=10), bm12→[B6→B6] (□, n=6), B6→[B6→II^{-/-}] (●, n=4), bm12→[B6→II^{-/-}] (▪, n=7). In another embodiment, GVHD was equally severe in [B6→II^{-/-}] recipients of bm12 T cells as in [B6→wt] recipients and all died by day 7, consistent with the equivalent donor CD4⁺ T cell activation and proliferation observed in these chimeras. In a further embodiment, clinical GVHD scores assessed on day 5 in [B6→II^{-/-}] recipients of bm12 donor cells (6.4±0.5) were equivalent to those of [B6→wt] recipients of bm12 T cells (6.4±0.1), and significantly greater than in both sets of two syngeneic controls (3.4±0.2, P<0.05).

GVHD pathology scores of the liver and small intestine in allogeneic [B6→wt] and [B6→II^{-/-}] recipients on day 5 post-BMT were significantly greater than those in corresponding syngeneic recipients (FIG. 9B, coded slides (n=4/group) were scored semi-quantitatively as described infra. B6→[B6→B6] (white bar), bm12→[B6→B6] (black bar), B6→[B6→II^{-/-}] (diagonal-hatching bar), bm12→[B6→II^{-/-}] (grey bar)). Liver histology showed standard histological features of acute GVHD included mild mononuclear cells infiltrates in bile ducts and portal triads, acidophilic bodies, and endothelialitis, which is characteristics of severe GVHD (Crawford, Graft-versus-host disease of the liver. in Graft-versus Host Disease, pp. 315-336 (Marcel Dekker, New York (1997)) (FIGS. 9C and 9D (arrowheads indicate endothelialitis of a hepatic vein and arrows indicate hepatocytes undergoing necrosis)). These changes were more prominent in [B6→II^{-/-}] recipients that lacked MHC class II expression on target cells (FIG. 9B). Small intestine histopathology also showed significant changes of GVHD in [B6→II^{-/-}] recipients of bm12 T cells, including villous atrophy with epithelial apoptosis (FIGS. 9E and 9F (arrowheads indicate apoptotic crypt cells)). Mice receiving 13 Gy TBI and no BM survived more than 10 days (data not shown), ruling out graft failure as a cause of this early mortality as described (Paris, et al., Science, 293:293-297 (2001)).

In another embodiment, the absence of a requirement for alloantigen on GVHD target cells was confirmd. A second set of chimeras [B6→bm12] were created in a similar fashion. In this particular embodiment, host target cells have MHC class II syngeneic to the bm12 donors but host APCs have MHC class II allogeneic to the donors. GVHD was equally severe in [B6→B6] and [B6→bm12] recipients of bm12 T cells and both groups died of GVHD by day 10 after BMT (FIG. 9G,B6→[B6→B6] (∘, n=6), bm12→[B6→B6] (□, n=6), bm12→[B6→bm12] (●, n=6). *P<0.05, **P<0.001 compared to corresponding syngeneic control), confirming the sufficiency of alloantigens on host APC alone to induce GVHD mortality.

In another embodiment of the invention, the survival of host APCs after BMT was examined. Six days after allogeneic BMT of bm12 donors (CD45.2⁺) into congeneic B6.Ly-5a (CD45.1⁺) recipients, flow cytometric analysis of splenocytes demonstrated that 1.1±0.1% of host DCs (defined as CD45.1⁺, CD11c⁺, CD3⁻, B220⁻, NK1.1⁻, Gr-1⁻) survived at this time. These results demonstrate that a small number of host DC still survived 6 days after allogeneic BMT. Effector T cell requires continued stimulation with antigens to survive (Sitkovsky, M. V. & Henkart, P. A. Mechanisms of T-cell-mediated cytotoxicity in vivo and in vitro. in Graft-vs-host disease, pp. 219-233 (Marcel Dekker, Inc., New York, (1997)).

While an understanding of the mechanism is not required to practice the present invention and the present invention is not limited to any particular mechanism, whether adoptively transferred, activated CD4⁺ T cells stimulated by alloantigens could cause GVHD in the absence of alloantigen was examined. bm12 mice were lethally irradiated and injected with 2×10⁶ T cells from B6 mice. Six days later, spleens were harvested and CD4+ T cells were negatively isolated by using CD8, DX5, MHC class II, CD11b, and CD11c MicroBeads and the autoMACS following Ficoll gradient centrifugation. 5×10⁶ CD4+ T cells were then transferred to lethally irradiated (13 Gy TBI) II−/− mice. Control II−/− recipients were injected with 5×106 CD4+ T cells isolated from naive bm12 mice. Seven days after transfer, GVHD was not observed in either group by all parameters tested including mortality, clinical score, weight change, and pathology (see Table. 1). This result suggested that alloantigen expression is needed on the small number of host APCs that survive radiation and that still reside in target organs or secondary lymphoid tissue which are critical to the continued activation of donor CD4⁺ T cells and the induction of acute GVHD.

TABLE 1
In vivo primed CD4⁺T cells fail to induce GVHD after
adoptive transfer to MHC II^{-/-} mice

| bm12 CD4+ | | Clinical | Weight | Pathology Scores | |
|---|---|---|---|---|---|
| T cells | Mortality | Scores | Change | Liver | Intestine |
| naive | 1/5 | 3.6 ± 0.1 | 27 ± 1% | 1.0 ± 0.6 | 6.5 ± 0.3 |
| in vivo | 0/5 | 3.1 ± 0.4 | 25 ± 4% | 1.4 ± 0.4 | 5.2 ± 0.5 |
| primed | | | | | |

### Neutralization of Inflammatory Cytokines Prevented GVHD in the Absence of Alloantigens on Target Epithelium

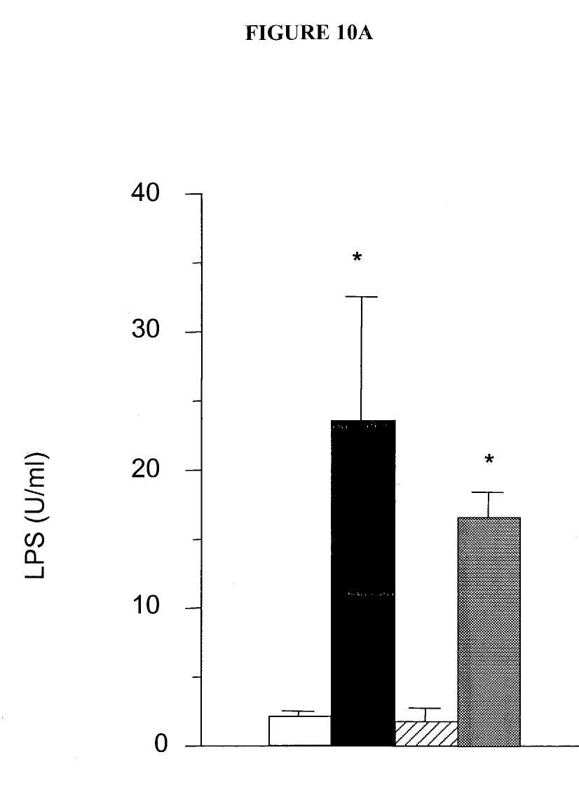

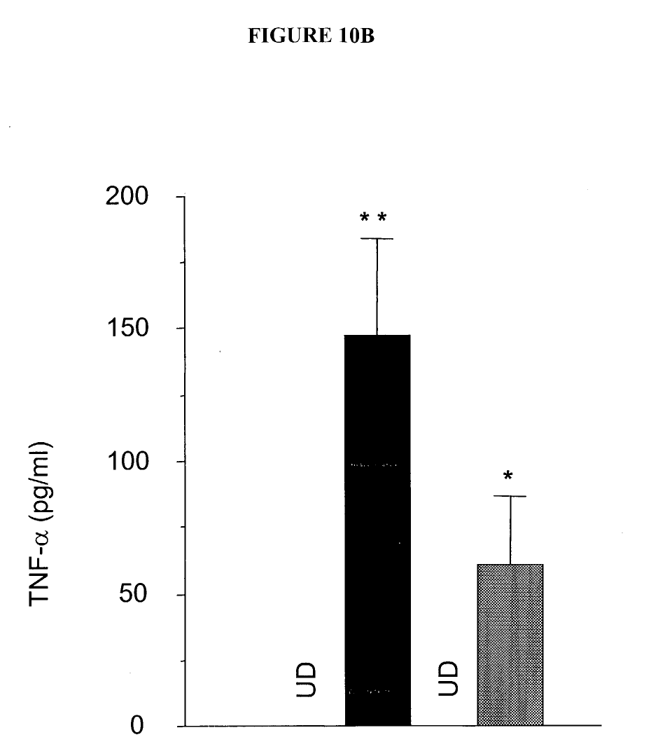

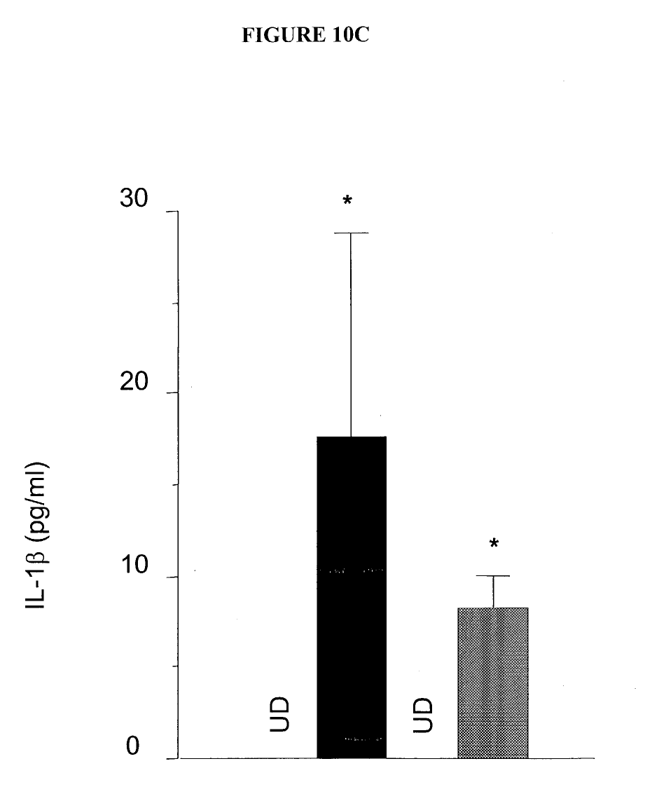

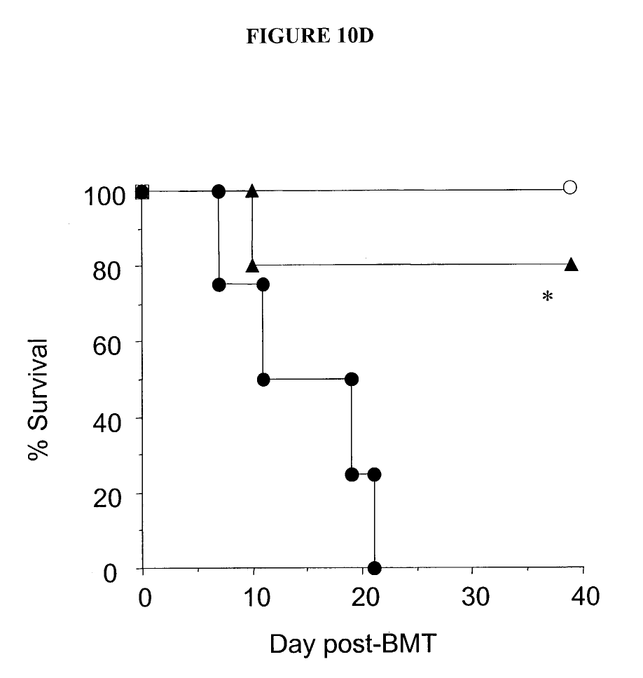

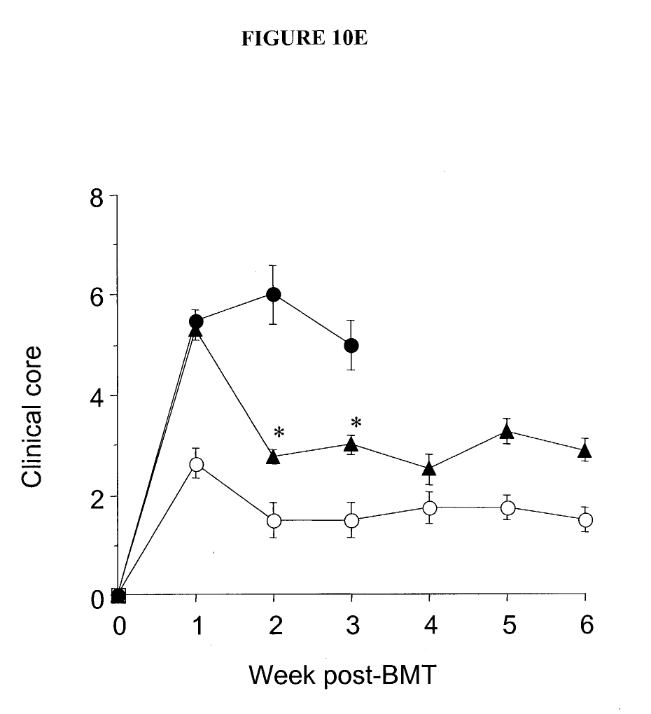

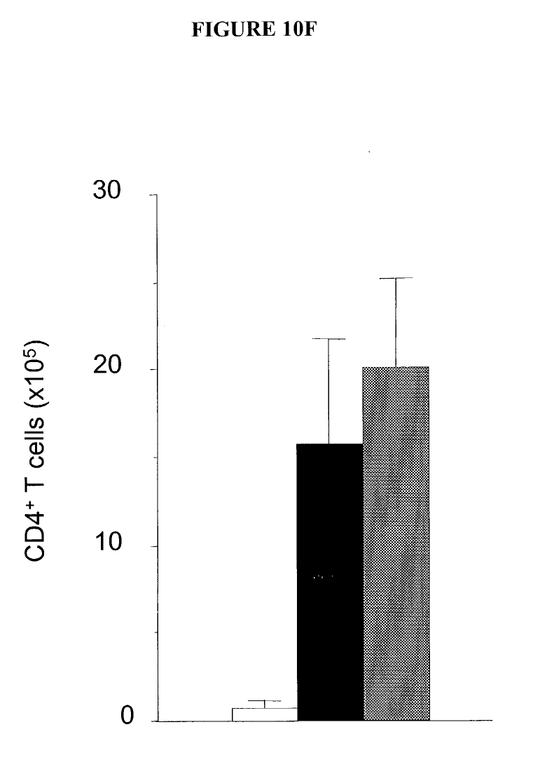

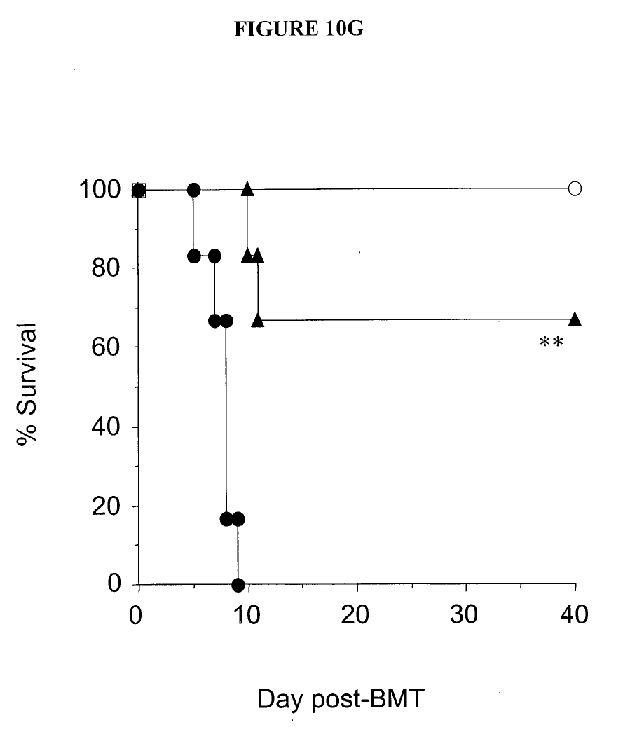

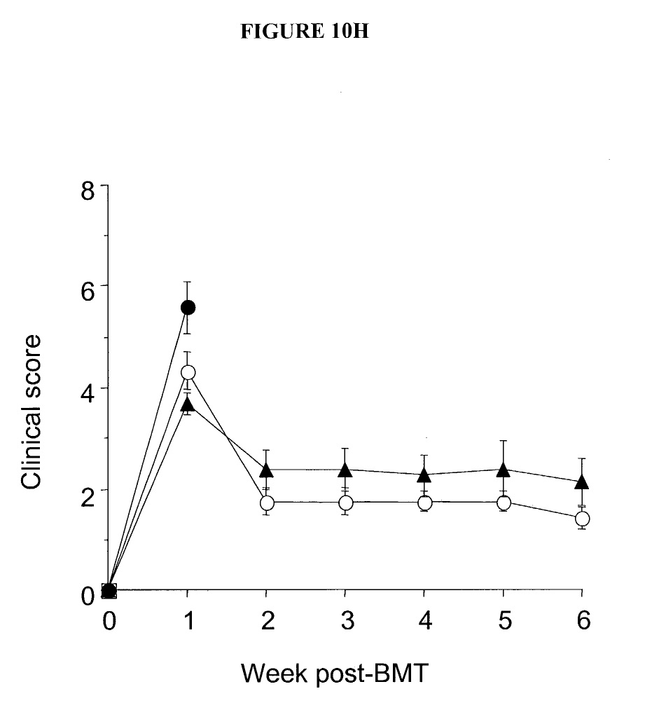

Inflammatory cytokines are produced during GVHD when mononuclear cells are primed by donor T cell IFN-γ and stimulated by lipopolysaccharide (LPS) (Hill et al., Blood, 90:3204-3213 (1997)). In an embodiment of the present invention, mice were transplanted as in FIG. 9 and described supra. Animals were killed on day 5 (n=3/group). Serum levels of LPS, which correlate well with the degree of small bowel injury (Hill et al., J. Clin. Invest., 104:459-467 (1999)) were equally high in [B6→II^{-/-}] recipients and [B6→B6] recipients of bm12 cells 5 days after BMT (see FIG. 10A, B6→[B6→II^{-/-}] (white bar), bm12→[B6→B6] (black bar), B6→[B6→II^{-/-}] (diagonal-hatched bar), bm12→[B6→II^{-/-}] (grey bar). Data represent mean ±SD. *P<0.05, **P<0.01 (bm12→[B6→B6] vs. B6→[B6→II−/−], B6→[B6→II^{-/-}] vs. bm12→[B6→II^{-/-}])). Serum levels of TNF-α and IL-1β were also equivalently elevated (see FIGS. 10B and 10C, UD=undetectable), demonstrating that host APCs can efficiently activate inflammatory effectors of GVHD in the absence of alloantigens on target cells.

While an understanding of the mechanism is not required to practice the present invention and the present invention is not limited to any particular mechanism, the production of inflammatory cytokines was examined to determine if they were related to GVHD mortality in this embodiment. BM chimeras were re-irradiated and injected with BM and T cells from either bm12 or B6 mice. TNF-α and IL-1 was neutralized with an i.p. injection of a combination of dimeric human soluble TNF-α receptor p80/IgG₁ Fc fusion protein (TNFR:Fc) and hamster anti-mouse type 1 IL-1 receptor (αIL-1R-M147) (Hill et al., J. Clin. Invest., 104:459-467 (1999)). None of [B6→II^{-/-}] recipients treated with control, which did not contain the cytokine inhibitor combination, survived; whereas 80% of mice treated with the cytokine inhibitors survived at day 40 (P<0.05, see FIG. 10D). Clinical GVHD scores of the surviving recipients were also significantly reduced in the group receiving the blockade of TNF-α and IL-1 (P<0.05, FIG. 10E, B6→[B6→B6] (, n=4), bm12→[B6→II^{-/-}] treated with diluent (●, n=4), bm12→[B6→II^{-/-}] treated with TNFR:Fc and αIL-1R (Δ, n=5)). Evaluation of donor T cells in spleens 5 days after GVHD induction in [B6→II^{-/-}] recipients showed by FACs analysis (n=3/group) that donor CD4⁺ T cell expansion was not suppressed by the cytokine blockade (see FIG. 10F, B6→[B6→B6] (white bar), bm12→[B6→II^{-/-}] treated with diluent (black bar), bm12→[B6→II^{-/-}] treated with TNFR:Fc and αIL-1R (grey bar)), confirming the effects of the blockade on the effector phase rather than activation phase of GVHD. The same combination of TNFR:Fc and αIL-1R improved survival and clinical GVHD scores in another embodiment ([B6→bm12]) (see FIGS. 10G and 10H, B6→[B6→B6] (, n=6), bm12→[B6→bm12] treated with diluent (●, n=6), bm12→[B6→bm12] treated with TNFR:Fc and αIL-1R (Δ, n=6). Δvs ●, *P<0.05, **P<0.01.) Importantly, clinical scores and weight loss of [B6→bm12] recipients of bm12 T cells were not statistically different from syngeneic controls, confirming that GVHD mortality is primarily mediated by inflammatory effectors without direct effector: target cell interactions or the expression of alloantigen on host target cells.

### CD8-Mediated Acute GVHD Does Not Require MHC Class I Alloantigen Expression on Host Epithelium

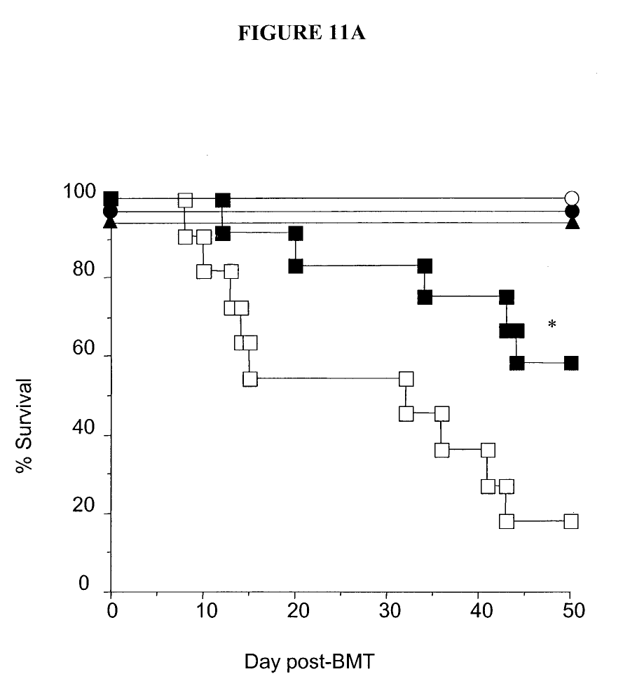

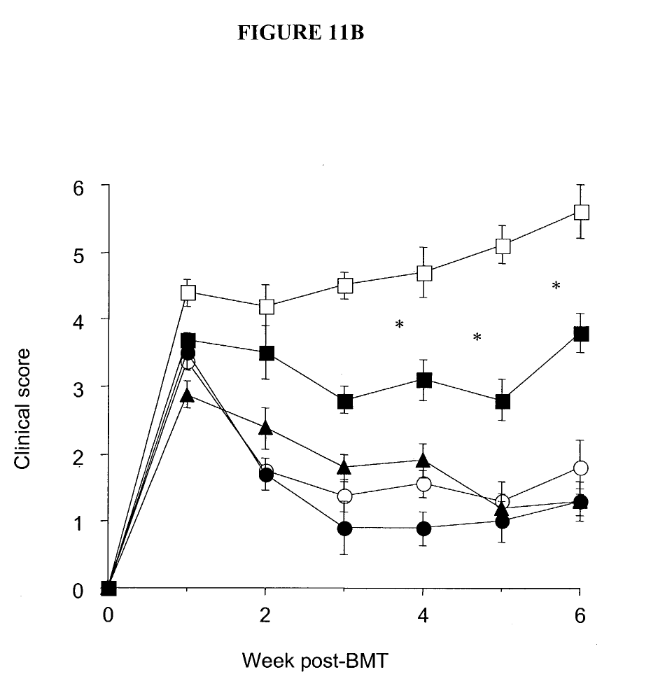

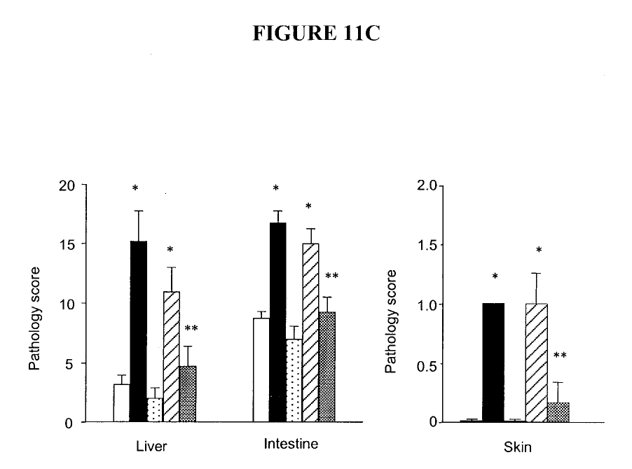

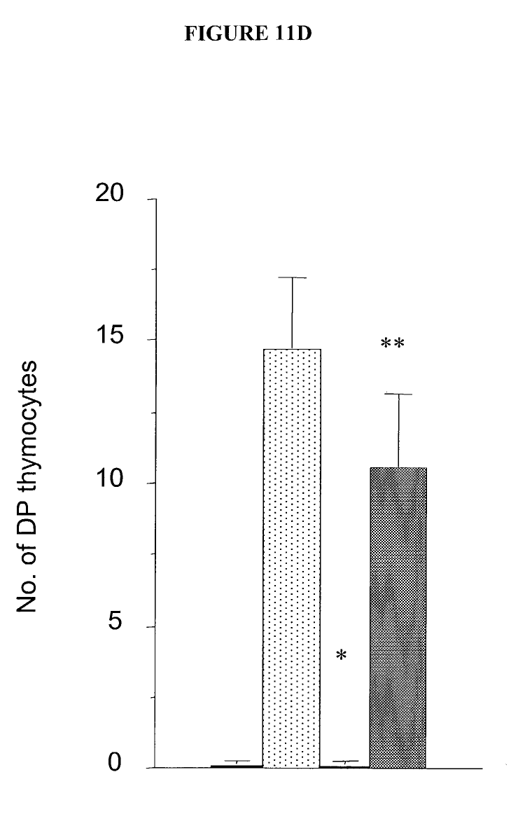

While an understanding of the mechanism is not required to practice the present invention and the present invention is not limited to any particular mechanism, whether MHC class I alloantigen expression on host target epithelium was similarly unnecessary for acute GVHD in a well characterized mouse model of GVHD directed to a single MHC class I alloantigen (bm1→B6) was examined. bm1 mice differ from B6 mice only by a mutation in the K^{b} class I antigen (Korngold and Sprent, T cell subsets in graft-vs.-host disease. in Graft-vs.-Host Disease: Immunology, Pathophysiology, and Treatment, pp.31-50 (Marcel Dekker, New York (1990).) In this donor-recipient strain combination, GVHD is mediated by CD8⁺ T cells and mortality is slower than in the bm12→B6 strain combination, as previously described (Shlomchik et al., Science, 285:412-415 (1999); Sprent et al., J. Exp. Med., 167:556-569 (1988)). In an embodiment of the present invention, [B6→bm1] and [B6→B6] chimeras were created by reconstituting lethally irradiated bm1 and B6 mice with 5×10⁶ T-cell depleted BM cells from B6 donors, respectively. Four months later, these chimeras were reirradiated with 13 Gy TBI and transplanted with 5×10⁶ TCD BM cells and 2×10⁶ CD8⁺ T cells from bm1 donors. bm1 CD8⁺ T cells produced 82% mortality by day 50 in [B6→B6] chimeras expressing allogeneic MHC class I on both APCs and target cells, whereas all [B6→B6] chimeric recipients of syngeneic B6 CD8⁺ T cells survived (see FIG. 11A). bm1 CD8⁺ T cells caused lethal GVHD in 42% of [B6→bm1] chimeric recipients that did not express MHC class I alloantigens on target epithelium, a significant decrease compared to 100% mortality in [B6→B6] recipients (see FIGS. 11A and 11B, B6→[B6→B6] treated with control (, n=9), bm1→[B6→B6] treated with control (, n=11), bm1 TCD BM→[B6→bm1] treated with control (●, n=6), bm1→[B6→bm1] treated with control (▪, n=12), bm1→[B6→bm1] treated with TNFR:Fc and αIL-1R (Δ, n=12). *p<0.05 (▪ vs □, Δ and ●)). Although mortality was delayed in [B6→bm1] chimeric recipients of bm1 CD8⁺ T cells, histological analysis was conducted, 45 days after BMT, of the liver, intestine (scores of intestine are the sum of those of small intestine and large intestine), and skin showed standard pathological features of acute GVHD (see FIG. 11C). (FIGS. 11C and 11D, B6→[B6→B6] (white bar), bm1→[B6→B6] (black bar), bm1 TCD BM→[B6→bm1] treated with control (speckled bar), bm1→[B6→bm1] treated with control (diagonal-hatched), bm1→[B6→bm1] treated with TNFR:Fc and αIL-1R (grey bar). *p<0.05 (bm1→[B6→B6] vs. B6→[B6→B6], bm1→[B6→bm1] treated with control vs. bm1 TCD BM→[B6→bm1] treated with control), **p<0.05 (bm1→[B6→bm1] treated with TNFR:Fc and αIL-1R vs. bm1→[B6→bm1] treated with control). Analysis of the thymus demonstrated equally reduced numbers of double positive thymocytes in both [B6→B6] and [B6→bm1] recipients of bm1 CD8⁺_T cells (see FIG. 11D). Thus the absence of MHC class I alloantigens on target epithelial cells significantly reduced the onset of acute GVHD mortality, but induced equally severe target organ damage in surviving mice. We next determined whether inflammatory cytokines were also effector molecules of acute GVHD in this CD8 dependent GVHD model. Neutralization of TNF-α and IL-1 with a combination of TNFR:Fc and αIL-1R from day −2 to day 35 again completely prevented acute GVHD by all parameters tested (mortality, clinical scores, and pathology) (FIG. 5).

### Experimental

The following examples are provided in order to demonstrate and further illustrate certain preferred embodiments and aspects of the present invention and are not to be construed as limiting the scope thereof.

In the experimental disclosure which follows, the following abbreviations apply: N (normal); M (molar); mM (millimolar); μM (micromolar); mol (moles); mmol (millimoles); μmol (micromoles); nmol (nanomoles); pmol (picomoles); g (grams); mg (milligrams); μg (micrograms); ng (nanograms); l or L (liters); ml (milliliters); μl (microliters); cm (centimeters); mm (millimeters); μm (micrometers); nm (nanometers); ° C. (degrees Centigrade); Gy (Grays); and total body irradiation (TBI)

### EXAMPLE 1

### Bone Marrow Transplant Mouse Model

In one study, female C57BL/6 (B6, H-2^{b}, CD45.2⁺), B6D2F1 (H-2^{b/d}, CD45.2⁺), B6SJLF1 (H-2^{b/s}), B10.BR (H-2^{k/k}), and CBA (H-2^{k/k}) mice were purchased from The Jackson Laboratory (Bar Harbor, Me., USA). B6.Ly5.2 (H-2^{b}, CD45.1⁺) mice were purchased from Frederick Cancer Research Facility (Frederick, Md., USA). Mice were transplanted according to a standard protocol described previously (Teshima et al., J. Clin. Invest. 104:317-325 (1999)). Briefly, mice received 7.5 or 11 Gy total body irradiation (TBI, ¹³⁷Cs source), split into two doses separated by 3 hours to minimize GI toxicity. BM cells (5×106) plus 2×106 nylon wool-purified splenic T cells from either allogeneic or syngeneic donors cells were resuspended in 0.25 ml of Leibovitz medium L-15 (GIBCO BRL; Life Technologies Inc., Carlsbad, Calif., USA) and injected intravenously into recipients on day 0. For engraftment experiments, B6.Ly5.2 (H-2^{b}, CD45.1⁺) animals were used as donors. In some cases, T-cell depletion of BM (using anti-Thy1.2 mAb and rabbit complement) was performed.

To create BM chimeras, young B6D2F1 mice (2 months old) received 11 Gy TBI to eradicate host hematopoiesis. BM cells (5×106) from young (2 months) or old (18 months) syngeneic mice were injected intravenously into recipients on day 0. After 3 months of hematopoietic engraftment and APC repopulation, these chimeric mice were used as recipients.

In another study, female C57BL/6 (B6, K^{b}D^{b}I-A^{b}I-E^{b}, CD45.2⁺), B6.Ly-5a (CD45.1⁺), B6.C—H2^{bm12} (bm12, K^{b}D^{b}I-A^{bm12}-I-E^{b}, CD45.2+), and B6.C—H2^{bm1} (bm1, K^{bm1}D^{b}I-A^{b}I-E^{b}, CD45.2⁺) mice were purchased from the Jackson Laboratories (Bar Harbor, Me.). bm12 and bm1 mice possess a mutant class II and class I allele that differs from B6 mice, respectively. B6-background MHC class II−/− mice 32 (B6.129-Abbtm1N5: CD45.2+) were purchased from Taconic (Germantown, N.Y.). The age range of mice used as BMT donor and recipients was between 9 and 16 weeks.

To create BM chimeras, mice received 13 Gy TBI (¹³⁷Cs source) split into two doses separated by 3 hours to minimize gastrointestinal toxicity, and then injected intravenously with 5×10⁶ T cell-depleted (TCD) BM cells from donor mice on day 0. TCD was performed by incubating BM cells with Thy-1.2 MicroBeads and the autoMACS (Miltenyi Biotec, Bergisch Gladbach, Germany) separation. Mice were housed in sterilized microisolator cages and received autoclaved hyperchlorinated drinking water for the first three weeks after BMT, and filtered water thereafter.

Four months after the generation of BM chimeras, GVHD was induced according to a standard protocol as described previously (Wall et al., J. Immunol., 140: 2970-2976. (1988)). For GVHD induction to MHC class II alloantigen, mice were reirradiated with 13 Gy TBI, split into two doses, and injected with 4×10⁶ nylon wool purified splenic T cells and 5×106 BM cells on day 0. For GVHD induction to MHC class I alloantigen, mice received 2×106 CD8+ splenic T cells with 5×106 CD4− and NK-depleted BM cells from bm1 donors after 13 Gy TBI. CD8+ T cells were negatively isolated by using CD4, DX5, MHC class II, and CD11b MicroBeads and the autoMACS following nylon wool purification of T cells from splenocytes. For analysis of cell division, splenic T cells were labeled with CFSE (Molecular Probes, Eugene, Oreg.) as described (Reddy et al., J. Exp. Med., 194:1433-1440 (2001)) and then transferred into irradiated recipients.

### EXAMPLE 2

### Clinical and Histologic Assessment of GVHD in Mice

In one study, survival was monitored daily, and GVHD clinical scores were assessed weekly by a scoring system incorporating five clinical parameters: weight loss, posture (hunching), activity, fur texture, and skin integrity, as described (Cooke et al., Blood, 88:3230-3239(1996)). Individual mice were ear-tagged and graded weekly on a scale from 0 to 2 for each criterion (maximum score 10). GVHD was also assessed by detailed histopathologic analysis of the small (ileum) and large (ascending) intestines using a semiquantitative scoring system as described previously (10).

In another study, survival after BMT was monitored daily and the degree of clinical GVHD was assessed weekly by a scoring system which sums changes in five clinical parameters: weight loss, posture, activity, fur texture, and skin integrity (maximum index=10) as described (Cooke et al., Blood, 88:3230-3239. (1996)). This score is a more sensitive index of GVHD severity than weight loss alone in multiple murine models (Cooke et al., Blood, 88:3230-3239. (1996)). Acute GVHD was also assessed by detailed histopathologic analysis of liver and intestine, two primary GVHD target organs as described (Cooke et al., Blood, 92:2571-2580 (1998); Hill et al., J. Clin. Invest., 102:115-123 (1998)). Slides were coded without reference to mouse type or prior treatment status and examined systematically by a single pathologist (C. L.) using a semiquantitative scoring system (Cooke et al., Blood, 92:2571-2580 (1998); Hill et al., J. Clin. Invest., 102:115-123 (1998)).

### EXAMPLE 3

### Cell Cultures

All culture media reagents were purchased from Life Technologies Inc. Cells were plated in 96-well flat-bottomed Falcon plates (Becton Dickinson and Co., Lincoln Park, N.J., USA) at a concentration of 2×10⁵ cells/well with 1×10⁵ irradiated (20 Gy) peritoneal cells lavaged from either naive B6D2F1 (allogeneic) or B6 (syngeneic) animals and maintained in a humidified atmosphere with 7.5% CO₂. Supernatants were collected after 48 hours for IL-2 and after 62 hours for IFN-γ measurement. Dendritic cells (DCs) were generated by culturing BM cells with 10 ng/ml GM-CSF and 10 ng/ml IL-4 at 1×10⁶ cells/ml (Inaba et al., J. Exp. Med., 176:1693-1702 (1992); Fields et al. J. Immunother. 21:323-339 (1998)). On day 5 of culture, DCs were enriched by density-gradient centrifugation using 14.5% metrizamide (Sigma Chemical Co., St. Louis, Mo., USA). DC fractions were removed from the low-density interface, washed twice, and incubated with anti-CD11c (N418) MicroBeads for magnetic cell separation with an AutoMACS system (Miltenyi Biotec, Bergisch Gladbach, Germany). The purity of the DC suspension was greater than 95% as determined by dual positivity for MHC class II and CD11c. For studies of TNF-α and IL-12 p40 secretion, 1×10⁵ DCs per well were plated in 96-well Falcon plates with 0.1 μg LPS/ml for 4 or 24 hours.

### EXAMPLE 4

### Cytokine ELISA

Concentrations of IFN-γ, IL-2, and IL-12 p40 were measured by sandwich ELISA as described (BD Pharmingen, San Diego, Calif., USA). (TNF-α) Quantikine; R&D Systems Inc., Minneapolis, Minn., USA) (Teshima et al., Cancer Res., 61:162-171 (2001)). To determine serum levels of LPS, the limulus amebocyte lysate (LAL) assay (BioWhittaker Inc., Walkersville, Md., USA) was performed according to the manufacturer's protocol. All units expressed are relative to US reference standard EC-6.

In a second study ELISA for IFN-γ, IL-1b, IL-2, IL-4, and TNF-α (BD Pharmingen) were performed as described (Teshima et al., Cancer Res., 61:162-171 (2001)). For determination of lipopolysaccharide (LPS) concentration in serum, the Limulus amebocyte lysate (LAL) assay (BioWhittaker, Walkersville, Md.) was performed as described (Hill et al., Blood, 90: 3204-3213 (1997)). Samples and standards were run in duplicate. Unit is relative to the U.S. reference standard EC-6.

### EXAMPLE 5

### Flow Automated Cytometric Analysis

FITC-, phycoerythrin- (PE-), and allophycocyanin-conjugated mAbs against mouse antigens (CD45.1, CD3ε, CD4, CD8, CD11b, CD11c (HL3), CD40, CD80, CD86, and I-A^{b}) were purchased from BD Pharmingen. The analysis was performed as described previously (Hill et al., Blood, 90:3204-3213(1997)). Briefly, cells were incubated with mAb 2.4G2 for 15 minutes at 4° C. and then with the relevant FITC-, PE-, or allophycocyanin-conjugated mAb for 30 minutes at 4° C. Cells were washed twice with 0.2% BSA in PBS and fixed with PBS and 1% paraformaldehyde. Three-color flow cytometry was performed with an EPICS Elite ESP cell sorter (Beckman Coulter Inc., Miami, Fla., USA). For analysis of cell division, splenic T cells were labeled with carboxyfluorescein succinimidyl ester (CFSE; Molecular Probes Inc., Eugene, Oreg., USA) as described (Reddy et al., J. Exp. Med., 194:1433-1440 (2001)) and then transferred into irradiated recipients. For intracellular cytokine staining, splenocytes were harvested 4 days after transplantation. Cells were incubated for 4 hours with PMA-ionomycin (BD Pharmingen) and brefeldin A at 37° C. Then cells were permeabilized with the Cytofix/Cytoperm Kit from Pharmingen (San Diego, Calif., USA) and subsequently stained with PE-conjugated IFN- mAb. Flow cytometry was conducted by gating for the designated cell populations.

In a second study, a flow cytometric analysis was performed using FITC-, PE-, or allophycocyanin (APC)-conjugated mAbs to mouse CD45.1, CD3ε, CD4, CD8α, CD11c (HL3), CD25, CD49, and I-A^{b} (BD Pharmingen, San Diego, Calif.). Cells were preincubated with 2.4G2 mAbs to block FcγR, and were then incubated with the relevant mAbs for 30 minutes at 4° C. Finally, cells were washed twice with 0.2% BSA in PBS, fixed with 1% paraformaldehyde in PBS and analyzed by EPICS Elite ESP cell sorter (Beckman-Coulter, Miami, Fla.). Irrelevant IgG2_{a/b} mAbs were used as a negative control. Ten thousand live events were acquired for analysis.

### EXAMPLE 5

### Splenic T Cell Depletion

CD4+ and CD8+ T cells were depleted using CD4− and CD8− MicroBeads and the AutoMACS system (Miltenyi Biotec) according to the manufacturer's protocol.

### EXAMPLE 6

### Statistical Analysis

In the first study, the Mann-Whitney U test was used for the statistical analysis of cytokine data, LPS levels, clinical GVHD scores, and histology, whereas the Cox regression test was used to analyze survival data. P<0.05 was considered statistically significant.

### EXAMPLE 7

### Administration of Etanercept for Treatment of GVHD

Acute GVHD (aGVHD) following bone marrow transplant is initially treated with solumedrol at a dose of 1 mg/kg/q 12 hours (2 mg/kg/day). Patients continue on solumedrol as well as cyclosporine, tacrolimus or other immunosuppressive drugs with appropriate serum levels for GVHD control. Solumedrol is maintained at a dose of 2 mg/kg/day for at least one week. Tapering is based on the patient's response to therapy.

Patients receive etanercept (ENBREL, Wyeth-Ayerst Laboratories) 25 mg (0.4 mg/kg for patients<0.6 m²) subcutaneously twice a week for the first eight weeks (maximum dose of etanercept=25 mg). Etanercept is initiated within 72 hours of starting solumedrol. No dose is administered within 72 hours of a prior dose. No premedication is required. All patients are observed for local reactions at the injection site within 30 minutes of the first dose.

At 8 weeks if the patient is in a CR and off solumedrol, the etanercept is administered for one more week and then stopped. If after 8 weeks of therapy the patient is on solumedrol but responding, the etanercept may be continued as long as solumedrol is continued or for a maximum of 16 weeks.

### Toxicity Grading

Toxicity of the administration of etanercept is measured as per the common NCI toxicity criteria. Any patient who develops ≧grade 3 toxicity attributed to etanercept during the first 8 weeks is discontinued from administration of the drug. Patients undergoing treatment of aGVHD often have multiple complications with grade 3 and 4 toxicity. As target organ deterioration (skin, liver and gut) is common in the treatment of aGVHD, every effort is made to determine the cause. If the clinical situation allows, re-biopsy of the organ is recommended to confirm the presence aGVHD. If repeat biopsy cannot be done and other causative factors are eliminated, deterioration is attributed to aGVHD. Any grade 3 or 4 non-target organ toxicity is examined for its relationship to the drug. Biopsy is recommended but not required. Patients are followed for adverse event reporting for 30 days after their final dose of etanercept.

### GVHD Assessment

Patients are seen at least twice weekly for the first four weeks, and then weekly for the next four. Formal GVHD grading as well as toxicity are done on days 0 and then weekly from the start of the treatment. Staging for GVHD is done for each organ; based on the staging, grading is done and used for the formal evaluation of response. The following labs are drawn on each evaluation including CBC with differential, chemistry to include bilirubin, AST, ALT, Alkaline phosphates, BUN and creatinine electrolytes and study bloods as outlined infra.

Once the initial diagnosis of GVHD is made, response is evaluated in each organ weekly. Specific organ involvement and staging is monitored weekly. Only changes in grade will be used to determine the response. As flares of GVHD are common during taper periods, the initial best response either a PR or CR is recorded as a positive outcome.

### Cytokine Analysis

Blood samples (15 ml's) are obtained from patients prior to initiation of treatment and then weekly during treatment. Blood is analyzed for plasma cytokine levels and for lymphocyte phenotype and cytokine expression. Plasma and single cell suspension mononuclear cells are frozen according to standard practice. Samples are batched and run at the same time in order to maximize efficiency. Plasma is analyzed for several cytokines such as TNF-α, soluble TNF Receptor (p55), IL-1β and IL-1 receptor antagonist levels. Cells are analyzed by an intracytoplasmic staining technique for TNF-α, IL-1β, IL-2, IL-4 and IFNγ. Quality control is ensured by comparisons with plasma and cells from normal volunteers with and without stimuli such as lipopolysaccharide as positive and negative controls, respectively. Prior to receiving the etanercept, plasma levels of soluble receptor (TNFR) and TNF-α are assayed using cytokine enzyme-linked immunosorbent assays (ELISA) that are commercially available from Genzyme (Cambridge, Mass.). The assays are performed according to the manufacturer's protocol. Recombinant soluble TNFR (Genzyme) are used as a standard for the ELISAs. Samples and standards are run in duplicate. Based on previous experience, the sensitivity of these assays is approximately 16-20 pg/mL depending on the dilution. There is a rise in levels of TNF-α, sTNFR or both at the onset of GVHD. Preclinical or clinical data predict that TNF-α and sTNFR level are higher in patients who develop GVHD than in those who do not, depending on whether or not there are other complications. Phenotyping of the lymphocytes for CD-3, CD-4, and CD-8 from these samples are performed by standard flow cytometry. The lymphocytes are co-cultured with irradiated recipient lymphocytes obtained pre-transplant and separately with concavalin A (ConA) for 48 hours in 96-well flat bottom plates. At 48 hours the supernatants are collected and assayed by ELISA as described above for IL-2, IL-4, and IFNγ using the appropriate commercially available monoclonal antibodies for the assays and commercially available recombinant IL-2, IL-4, and IFN-γ for control assays. The sensitivity of the assay is 0.5 U/mL, which is more than adequate based on the animal studies. Pro-inflammatory cytokine production by lymphocytes are evaluated both prior to and after etanercept and steroid treatment and these findings with clinical response are correlated

### EXAMPLE 8

### Treatment for Patients with Acute Lung Injury Post Transplant: Idiopathic Pneumonia Syndrome (IPS)

Patients undergo a single bronchoalveolar lavage (BAL) or lung biopsy at baseline, prior to initiation of etanercept therapy. For patients with IPS, the bronchoalveolar lavage is performed 24-48 hours prior to the administration of the first dose of etanercept. If the bronchoscopy or lung biopsy are positive for infectious pathogens, then the patient may not receive therapy with etanercept at that time. A total volume of 2 mg/kg of sterile NS (max volume 180 cc) is instilled into the patient's lungs. When possible, BAL samples are collected from both the right and left sided airways. After BAL fluid collection, BAL fluid samples are pooled and then divided and distributed for the following assays: cell count and cytospin; stains for bacteria, fungi, AFB and PCP; quantitative culture for bacteria; fungal, mycobacterial and viral cultures; PCR for PCP; cytology; VNTR analysis of donor-host hematopoietic chimerism; BAL fluid cytokine analysis; BAL fluid chemokine analysis; and BAL cell pellet for mRNA extraction.

BAL fluid are analyzed for the cytokines and inflammatory markers. The BAL fluid is placed in a sterile container, and kept refrigerated at 4° C.

Transbronchial biopsy procedure may be performed at the discretion of the treating physician. If transbronchial biopsy is performed, then tissue is sent for histopathology and for micro-organism culture as described supra.

### Corticosteroids

Patients receive methylprednisolone at 2 mg/kg/day or equivalent. Steroids may be tapered as per our standard practice guidelines for acute GVHD therapy.

### Etanercept Dose

In some embodiments, 0.4 mg/kg/dose to a maximum single dose of 25 mg, subcutaneously, twice weekly, for a total of 8 dosages are given. In some embodiments, no dose is administered within 72 hours of a prior dose. No pre-medication is required. All patients are observed for local reactions at the injection site within 30 minutes of the dose.

### Schedule of Etanercept Administration

During the first week of therapy, BAL fluid analysis is sent at the start of treatement. Between 24 and 48 hours post-BAL, the first dose of etanercept is administered. 72 hours after the first dose, the second does of etanercept is administered. For the following three weeks, etanercept is given twice per week with 72 to 96 hours between doses.

### Observations (IPS Patients): Week 1 to 4 of Therapy

The following observations and assays are made between weeks 1 and 4 of the therapy. Blood and serologic tests including CBC with differential and platelet count (2×/wk); serum electrolytes, BUN/creatinine, liver panel, albumin (2×/wk) CMV testing via PCR, CMV pp65, or viral load by hybrid capture analysis (weekly), serum cytokine studies (weekly). Radiographic tests including CXR 2×/wk (week 1) then weekly (week 2-4), chest CT scan, if performed initially, should be repeated at week 4. Pulmonary function analysis including room air oxygen saturation (spO2) (daily); FiO2 required to spO2>93% recorded (daily); if patient is intubated and has an arterial catheter in place A-a gradient as assessed by ABG (2×/wk); if on mechanical ventilation: PaO2/FiO2 ratio (2×/wk); if on mechanical ventilation: (MAP×FiO2)/PaO2 ratio (2×/wk); if on mechanical ventilation, record lung compliance and airflow resistance measurements (2×/wk); if on mechanical ventilation: record total time on mechanical ventilation; if not on mechanical ventilation, and patient 6 years and can comply with instructions: pulmonary function testing including FVC, FEV1.0/FVC and DLCO measurements (day 28). Acute Graft Versus Host Disease grading conducted weekly. Repeat BAL procedure on day 28 and test fluid for cell count, cytospin, molecular diagnostics and cytokine/chemokine analysis; if intubated, repeat BAL procedure on day 14 and send fluid for cell count, cytospin, molecular diagnostics and cytokine/chemokine analysis.

### Observations (IPS Patients): Week 8 of Therapy (Day 56)

Blood tests including CBC with differential and platelet, serum electrolytes, BUN, creatinine, liver panel, albumin CMV serum cytokine analysis are conducted. Pulmonary function Analysis are also conducted which include room air oxygen saturation (spO2); FiO2 required to maintain spO2>93%; if not on mechanical ventilation, and patient >6 years, pulmonary function testing for FVC, FEV1.0/FVC, DLCO; if still on mechanical ventilation, repeat PaO2/FiO2 ratio (2×/wk) and MAP×FiO2)/PaO2 ratio (2×/wk). Conduct acute Graft Versus Host Disease grading.

### Observations: Month 4

The following observations are conducted at month 4: clinic visit; clinical status and interim history; pulmonary function analyses; room air oxygen saturation (spO2); and if patient is >6 years old and able to comply, pulmonary function testing including FVC, FEV1.0/FVC, TLC, and DLCO.

### EXAMPLE 9

### Treatment for Patients with Sub-Acute Pulmonary Dysfunction Following Bone Marrow Transplantation

### Pretreatment

The following tests are conducted within 14 days prior to treatment. Blood and serologic tests, which include, CBC with differential and platelets, serum electrolytes, BUN/creatinine, liver panel, albumin serum cytokine measurements (2 green top tubes), serum quantitative immunoglobulin levels, serum β-HCG test if patient is a female of child bearing age. In addition, tests for infectious diseases are also conducted which include tests for CMV pp65 antigen and urine analysis for adenovirus. Radiographic studies are also performed, which include CXR (PA/lateral or portable AP) and high-resolution chest CT. Pulmonary function testing includes room air oxygen saturation (SpO2); if the patients is 6 years of age, and is able to comply with instructions:FVC, FEV1.0/FVC, TLC, DLCO; and FiO2 required to keep SpO2>93%. Also, chronic graft-versus host disease assessment is also conducted.

### Bronchoalveolar Lavage (BAL)

Patients undergo BAL prior to initiation of etanercept therapy. For patients with subacute lung injury, the BAL is performed no more than 2 weeks prior to initiation of etanercept therapy.

### Bronchoalveolar Lavage (BAL) Procedure

A total volume of 2 mg/kg of sterile NS (max volume 180 cc) is instilled into the patient's lungs. When possible, samples are collected from both the right and left sided airways. After collection, BAL fluid samples are pooled and then divided and distributed for the following assays: cell count and cytospin; stains for bacteria, fungi, AFB and PCP; quantitative culture for bacteria; fungal, mycobacterial and viral culture, PCR for PCP; cytology; vntr analysis of donor host hematopoietic chimerism; BAL fluid cytokine analysis; BAL fluid chemokine analysis; BAL cell pellet for mRNA extraction. BAL fluid will be analyzed for the cytokines and inflammatory markers. The BAL fluid is placed in a sterile container, and kept refrigerated at 4° C.

### Transbronchial Biopsy Procedure

At the discretion of the treating physician, if a transbronchial biopsy is performed, then tissue is sent for histopathology and for micro-organism culture.

### Corticosteroid Therapy

Patients with chronic graft versus host disease currently on corticosteroid therapy are to continue their corticosteroids without dose alteration during the 4 week course of Etanercept therapy.

### Etanercept Therapy for Patients with Sub-acute Lung Injury

Etanercept therapy is initiated after the following: at least 24 hours have elapsed from time of completion of the BAL procedure; BAL fluid special stains, including the gram stain, fungal stain, AFB stains that are negative at 24 hours; BAL fluid quantitative bacterial culture (>10⁴ CFU/ml is considered positive), fungal, AFB and viral culture with negative results at 24 hours.

### Etanercept Dose

In some embodiments, 0.4 mg/kg/dose to a maximum of 25 mg is administered subcutaneously, twice weekly, for a total of 8 dosages. In some embodiments, no dose is administered within 72 hours of a prior dose. No pre-medication is required. All patients are observed for local reactions at the injection site within 30 minutes of the first dose.

### Schedule of Etanercept Administration

During the first week of therapy, BAL fluid analysis is sent at the start of treatement. After 24 hours has elapsed post-BAL, the first dose of etanercept is administered. The second dose of etanercept is administered between 96 and 120 hours after the first dose. For the following three weeks, etanercept is given twice per week with 72 to 96 hours between doses. The etanercept is administered Monday & Thursdays, or Tuesdays and Fridays.

### Observations: Week 1-4 (Day 1-28)

Blood and serologic tests are conducted, which include CBC with differential and platelet count (done weekly); serum electrolytes, BUN, creatinine, liver panel, albumin (done weekly); and serum cytokine studies(done week 4). In addition, radiographic studies are conducted, which include CXR and chest CT, these tests are repeated at week 4 if results are abnormal initially. Also, pulmonary function analyses are conducted, which include room air oxygen saturation (spO2) (weekly); FiO2 required to spO2>93% (weekly); if patients are 6 years: pulmonary function testing including FVC, EV1.0/FVC, TLC, DLCO (week 4). Finally, the BAL procedure is repeated within one week of completion of etanercept therapy (following 8^{th} dose). Fluids are analyzed for cell count, cytospin, molecular diagnostics and cytokine/chemokine analysis as supra.

### Observations: Week 8 (Day 56)

During Week 8, a clinical visit is conducted and clinical status and interim history is recorded. An infection history is also taken with determination of cGVHD status. In addition, blood tests are conducted which include CBC with differential and platelet; serum electrolytes, BUN, creatinine, liver panel, albumin; and serum cytokine analysis. Pulmonary function analyses are conducted as well, which include: room air oxygen saturation (spO2) (done weekly); FiO2 to keep spO2>93%; and patients who are 6 years old and able to comply, pulmonary function testing also includes FVC, FEV1.0/FVC, TLC, and DLCO.

### Observations: Month 2-12

During Months 2-12 the following observations and determinations are made: monthly clinical visits, clinical status and interim history is taken, infection history is taken and cGVHD status is determined. In addition, blood tests are conducted with each clinical visit, which include CBC with differential and platelet as well as serum electrolytes, BUN, creatinine, liver panel, and albumin tests. The following pulmonary function analyses are conducted as well, room air oxygen saturation (spO2) with each clinic visit; FiO2 to keep spO2>93% (with each clinic visit); and if the patient is >6 years old and able to comply, pulmonary function testing include FVC, FEV1.0/FVC, TLC, DLCO (q 2 months×5) as well.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A method of transplanting hematopoietic stem cells in a subject diagnosed with a hematopoietic disease comprising the steps of:
a) providing:
i) a subject diagnosed with a hematopoietic disease,
ii) hematopoietic stem cells,
iii) an agent that inhibits TNFα, and
iv) an agent that inhibits IL-1;
b) transplanting said hematopoietic stem cells into said subject to produce a hematopoietic transplant subject; and
c) administering said agent that inhibits TNFα and said agent that inhibits IL-1 to said hematopoietic transplant subject.

2. The method of claim 1, wherein prior to said transplanting step, said subject is irradiated with a myeloablative dose of radiation.

3. The method of claim 1, further comprising administering said agent that inhibits TNFα and said agent that inhibits IL-1 to said subject prior to transplanting said hematopoietic stem cells.

4. The method of claim 1, wherein said hematopoietic disease comprises a hematopoietic malignancy selected from the group consisting of leukemia, myelodysplastic syndrome, lymphoma, and plasma cell dyscrasia.

5. The method of claim 1, wherein said hematopoietic stem cells are allogeneic hematopoietic stem cells.

6. The method of claim 5, wherein said allogeneic hematopoietic stem cells are from a donor related to said subject.

7. The method of claim 1, wherein said hematopoietic stem cells are selected from the group consisting of bone marrow stem cells, peripheral blood stem cells and umbilical cord blood stem cells.

8. The method of claim 1, wherein said agent that inhibits TNFα comprises a recombinant soluble TNF receptor.

9. The method of claim 1, wherein said agent that inhibits IL-1 comprises an IL-1R-reactive antibody.

10. A method of treating acute graft versus host disease, comprising the steps of:
a) providing:
i) a subject with acute graft versus host disease,
ii) an agent that inhibits TNFα, and
iii) an agent that inhibits IL-1;
b) administering said agent that inhibits TNFα and said agent that inhibits IL-1 to said subject with acute graft versus host disease.

11. The method of claim 10, wherein said administering comprises a regimen effective for reducing serum TNFα levels of said subject.

12. The method of claim 10, wherein said administering comprises a regimen effective for reducing serum IL-1β levels of said subject.

13. The method of claim 10, wherein said administering comprises a regimen effective for increasing the length of post transplant survival of said subject.

14. The method of claim 10, wherein said administering comprises a regimen effective for reducing clinical graft versus host disease grade.

15. The method of claim 10, wherein said administering comprises a regimen effective for reducing skin pathology of said subject.

16. The method of claim 15, wherein said reducing skin pathology comprises maculopapular rash reduction.

17. The method of claim 10, wherein said administering comprises a regimen effective for reducing liver pathology of said subject.

18. The method of claim 17, wherein said reducing liver pathology comprises reducing elevated serum bilirubin levels.

19. The method of claim 10, wherein said administering comprises a regimen effective for reducing intestinal pathology of said subject.

20. The method of claim 19, wherein said reducing intestinal pathology comprises reducing diarrhea.

21. A method of treating pulmonary dysfunction occurring after allogeneic stem cell transplantation, comprising the steps of:
a) providing:
i) a subject diagnosed with pulmonary dysfunction, wherein said pulmonary dysfunction is associated with prior allogeneic stem cell transplantation,
ii) an agent that inhibits TNFα, and
iii) an agent that inhibits IL-1; and
b) administering said agent that inhibits TNFα and said agent that inhibits IL-1 to said subject diagnosed with pulmonary dysfunction.

22. The method of claim 21, wherein said pulmonary dysfunction is the result of a noninfectious lung injury.

23. The method of claim 22, wherein said pulmonary dysfunction comprises a disease selected from the group consisting of bronchiolitis obliterans, restrictive lung disease and idiopathic pneumonia syndrome.

24. The method of claim 21, wherein said agent that inhibits TNFα comprises a recombinant soluble TNF receptor.

25. The method of claim 21, wherein said agent that inhibits IL-1 comprises an IL-1R-reactive antibody.

26. The method of claim 20, wherein said administering comprises a regimen effective for improving the results of at least one pulmonary function test of said subject.

27. The method of claim 26, wherein said at least one pulmonary function test comprises a test selected from the group consisting of a forced vital capacity test (FVC), a forced expiratory volume in one second test (FEV_{1.0}) and a diffuse capacity of lungs for carbon monoxide test (DLCO).

28. A pharmaceutical preparation comprising an agent that inhibits TNFα, an agent that inhibits IL-1, and instructions or labels for using said pharmaceutical preparation to treat or prevent conditions associated with stem cell transplantation.
